(19) United States
(12) Patent Application Publication
(10) Pub. No.: US 2010/0227876 A1
(43) Pub. Date: Sep. 9, 2010

(54) Methods of Reducing Side Effects of Analgesics

(75) Inventor: Richard H. Rech, Okemos, MI (US)

Correspondence Address: MARSHALL, GERSTEIN & BORUN LLP, 233 SOUTH WACKER DRIVE, 6300 WILLIS TOWER, CHICAGO, IL, 60606-6357, US

(73) Assignee: RECHFENSEN LLP, Ridgewood, NJ (US)

(21) Appl. No.: 12/399,629

(22) Filed: Mar. 6, 2009

(51) Int. Cl. A61K 31/485; A61K 31/40; A61K 31/445; A61K 31/439

(52) U.S. Cl. 514/282; 514/409; 514/329

(57) ABSTRACT

The invention provides for compositions and methods of reducing pain in a subject by administering a combination of mu-opioid receptor agonist, kappa1-opioid receptor agonist and a nonselective opioid receptor antagonist in amounts effective to reduce pain and ameliorate an adverse side effect of treatment combining opioid-receptor agonists. The invention also provides for methods of enhancing an analgesic effect of treatment with an opioid-receptor agonist in a subject suffering from pain while reducing an adverse side effect of the treatment. The invention also provides for methods of reducing the hyperalgesic effect of treatment with an opioid-receptor agonist in a subject suffering from pain while reducing an adverse side effect of the treatment. The invention further provides for methods of promoting the additive analgesia of pain treatment with an opioid-receptor agonist in a subject in need while reducing an adverse side effect of the treatment.

### FIELD OF INVENTION

The invention provides for compositions of opioid-receptor agonists and opioid-receptor antagonists and methods for reducing pain in subjects.

### BACKGROUND

In attempts to develop analgesics devoid of mu-opioid receptor type tolerance, dependence, opioid-induced hyperalgesia (OIH), and addiction lability, kappa-opioid receptor agonists were developed (Walker et al., Psychopharmacology. 155: 362-71 (2001); Wadenberg, CNS Drug Rev. 9(2):187-98, 2003)). Pentazocine, the first kappa-opioid receptor agonist marketed (a mixed agonist, with mu-opioid receptor activity) had little effect on respiratory function, limited analgesic efficacy and low dependence/addiction liability. But as a partical mixed agonist pentazocine also had modest efficacy at kappa1-and kappa2-receptors. Unpleasant side effects of anxiety, dysphoria, and psychotomimetic actions hindered patient acceptance.

Henck et al. (Pharmacol Biochem Behav. 18(1):41-5.1983)) studied a congener, cyclazocine, reporting a behavioral spectrum in rats similar to the hallucinogens lysergic acid diethylamide (LSD), 1-(2,5-Dimethoxy-4-methylphenyl)-2-aminopropane (DOM), mescaline, and dimethyltryptamine (DMT), and these actions are mediated via brain 5-HT-2 agonist activity. Additional kappa-opioid receptor agonists were shortly introduced: butorphanol (mixed partial agonist at kappa1-, kappa2-, and mu-opioid receptors) and nalbuphine (kappa1-, kappa2-receptor agonist and mu-opioid receptor antagonist), with pentazocine-like side effects. Nevertheless, butorphanol has had more success for pain relief in human and veterinary medicine than pentazocine, perhaps relating to the more definitive mixed agonist efficacies.

A host of research studies have demonstrated prominent antinociceptive effects of kappa-opioid receptor agonists over the last three decades, particularly for the arylacetamide class (U-50,488H, enadoline, spiradoline, U-69, 593). Using the colorectal distension assay (CRD, a model of visceral pain) in dogs, Sawyer et al. (Amer. Vet. Res. J. 52: 1826-30 (1991)) reported enhanced antinociception by butorphanol when combined with oxymorphone or ketamine. Ketamine attenuates acute mu-opioid tolerance by antagonizing NMDA activity and thus inhibits emergence of endogenous excitatory opioid receptor systems, decreasing their activation of pain-facilitatory systems (Fundytus, CNS Drugs. 15: 29-85 (2001)). Briggs et al. (Vet. Surg. 27: 466-72 (1998)) combined oxymorphone and butorphanol in cats tested in CRD to enhance antinociception and reduce side effects, as had been noted in dogs by Houghton et al., Proc. Soc. Exp. Biol. Med. 197: 290-6 (1991).

Side effects of mu-opioid receptor agonists (euphoria, constipation, enuresis, pruritus) are often mirror images of those of kappa-opioid receptor agonists (dysphoria, minor gastrointestinal effects, diuresis, anti-pruritus) in a variety of mammalian species (Pasternak et al., Life Sci. 138: 1889-98 (1986). Thus, the combination of mu- and kappa-opioid receptor agonists tends to reciprocally reduce each agonists' side effects, while producing additive antinociception in the cold-water tail-flick assay (CWTF). Briggs, Interactions of mu and kappa opioid agonists. Ph.D. Thesis Dissertation (1996), Briggs et al. Pharmacol. Biochem. Behav. 60: 467-72 (1998). Briggs et al., Pharmacol. Biochem. Behav. 60: 467-72 (1998) also demonstrated selective antagonism of mu-opioid receptor antinociception by beta-funaltrexamine (β-FNA) and selective antagonism of kappa-opioid receptor antinociception by nor-binaltorphamine (n-BNI) in the CWTF model.

Additive or enhanced pain relief, with reduced side effects produced by combining agonists, were also observed by Verborgh et al., Acta. Anaesthesiol. Scand. 41: 895-902 (1997).; Sutters et al., Brain Res. 530: 290-4 (1990); and Ross et al., Pain. 84: 421-8 (2000), among others. Bie et al., J. Neurosci. 23: 7262-8 (2003), on the other hand, reported potent antagonism of mu-opioid receptor agonist analgesia by kappa-opioid receptor agonists with acute dosing targeted at the brainstem nucleus raphe magnus. But they also showed that kappa-opioid receptor agonists reversed the hyperalgesia induced by chronic mu-opioid receptor agonist activation. Thus, the interactions of these agonists differed on the basis of acute and chronic treatment. They also appear to differ on the basis of single-site application versus diffuse application, as with systemic administrations or mixed administrations of mu-opioid receptor agonists microinjected into brain sites and kappa agonists applied to spinal sites by intrathecal injection (Miaskowski et al., Brain Res. 608: 87-94 (1993).

The work of Yaksh (Acta. Anaesthesiol. Scand. 41: 94-111 (1997)) relates in large part to concepts of visceral pain and opioid actions on visceral receptors. Yaksh used microinjections of drugs into supra-spinal (brain) and spinal sites to test effects on chemical, mechanical, or high-level thermal nociceptive stimuli. These antinociceptive insults preferentially activate visceral pain. Yaksh and colleagues formulated theories of complex pain pathways and drug actions still extant today. This work proposes that ascending and descending neuronal circuits connect to brain stem hubs (periaqueductal gray (PAG), mu-opioid receptors; rostral-ventral medulla, mu/delta-opioid receptors; substantia nigra, mu-opioid receptors) and to the spinal and dorsal-horn junctions (mu-/delta-/kappa-opioid receptors). Yaksh described these pathways and junctions as integrating and modulating nociceptive and antinociceptive impulses and greatly elucidated the concepts of mechanisms of pain perception and analgesic drug actions.

Use of combinations of mu- and kappa-opioid receptor agonists has been proposed to improve therapy of chronic clinical pain (Smith, Pain Physician. 11: 201-14 (2008)). This hypothesis portended separate antinociceptive drug actions on junctions in parallel- or serially-connected neuronal chains in pain-related central nervous regions as affecting synergistic analgesic interactions. Chronic visceral pain, more often prevalent in severe clinical cases than is somatic and cutaneous pain, is also more difficult to manage (Joshi et al., Curr. Rev. Pain. 4: 499-506 (2000). This relationship related to Smith's hypothesis, since visceral pain is relieved consistently with mu- or kappa-opioid receptor agonists alone or in combination (Ness et al., Pain. 41: 167-234 (1990); von Voigtlander et al., J. Pharmacol. Exp. Ther. 246: 259-62 (1988); Miaskowski et al. Brain Res. 608: 87-94 (1993), Briggs et al., Pharmacol. Biochem. Behav. 60: 467-72 (1998)). Also of interest in this sphere is the large number of arylacetamide kappa-opioid agonist class of drugs demonstrating selective, highly efficacious, and full-range of antinociceptive effects (see Briggs et al. Pharmacol Biochem Behav. 60: 467-72, 1998)).

Morphine and other mu-opioid receptor agonists are not suitable as analgesics in feline species because they produce manic excitation. This result is due to the fact that the nervous systems of cats contain dominant excitatory mu-opioid receptors, whereas in humans, monkeys and dogs the inhibitory opioid systems are dominant. Therefore Sawyer et al. (J. Amer. Hosp. Assoc. 23: 438-46 (1987)) tested butorphanol for analgesic activity in domestic cats against experimental visceral pain induced by the colorectal distension assay (CRD). Effective antinociception was obtained, the cats initially showing calmness, purring, and kneading. But as the analgesic response waned, the animals became somewhat irritable. These results suggested that the initial drug response reflected both mu- and kappa-opioid receptor analgesic effects, while the later responses, as the mu-opioid receptor agonism declined, reflected unopposed kappa-opioid receptor agonistic effects.

Sawyer et al. (Amer. Vet. Res. J. 52: 1826-30 (1991)) and Houghton et al. (Proc. Soc. Exp. Biol. Med. 197: 290-6 (1991)) also studied butorphanol analgesia against CRD in dogs. Effective analgesia was produced and was enhanced by addition of oxymorphone (mu-opioid receptor agonist), with reduced respiratory depression relative to the degree of oxymorphone respiratory depression when oxymorphone was administered alone.

Studies using combinations of fentanyl (mu-opioid receptor agonist) and enadoline or spirodoline antinociceptive doses were performed in place preference (PP) vs. place aversion (PA) conditioning procedures in rats (Briggs, Interactions of mu and kappa opioid agonists. Ph.D. Thesis Dissertation (1996); Briggs et al., Pharmacol. Biochem. Behav. 60: 467-72 (1998)). The conditioned place preference of fentanyl in a X-maze or a black-white two compartment maze was attenuated by combinations with enadoline or spiradoline. Of special interest was the finding that training and testing of subjects with spiradoline showed significant dose-related PA that was attenuated when the drug was combined with fentanyl.

Treatment of chronic pain with mu- or kappa-opioid agonist leads to development of tolerance and dependence, in large measure by activation of endogenous excitatory opioid systems. Mu-opioid agonists in particular promote opioid-induced hyperalgesia (OIH). Both kappa-opioid agonists and ultra-low doses of nonselective antagonists (naloxone, naltrexone) suppress mu-opioid receptor agonist induced OIH and dependence characteristics, reinstating and prolonging the analgesia of low doses of mu-opioid agonists. The ultra-low antagonist doses block activation of excitatory opioid systems but do not affect the activation of inhibitory opioid systems. Furthermore, the addiction liability of mu-opioid agonists (reward effect) is suppressed by the opposing mood effect (aversion) of kappa-opioid agonists. The combination with kappa-opioid agonists plus ultra-low dose antagonists also reduces tolerance and dependence of the mu-opioid agonists, thereby enhancing analgesia and decreasing compulsive mu-opioid agonist abuse. Therefore, there is a need to develop pain management treatments and opioid receptor agonist/antagonist compositions that effectively reduce or suppress pain while the emergence of the adverse side effects is also reduced.

### SUMMARY OF INVENTION

The invention provides for methods of reducing pain and adverse side effects in a subject comprising the administration of the combination of three opioid classes of drugs: (1) A mu-opioid receptor agonist (e.g. fentanyl, oxymorphone) at a moderate dose level such as the ED-50 dose or less; (2) a kappa-opioid receptor agonist (e.g. spiradoline, enadoline, U69593) at a moderate dose level such as the ED-50 dose or less; (3) a nonselective opioid receptor antagonist (e.g. naloxone, naltrexone) at an ultra-low dose level (such as a nanogram dose level) that suppresses tolerance and dependence development in both classes of agonists. The combined agonists afford additive or synergistic analgesia, depending on the type of pain involved, and greater pain relief at lower dose levels than when the agonists are administered singly. The incidence and intensity of adverse side effects are less by virtue of the lower agonist dose levels, as well as by interactions of the opposing spectra of agonists' side effects. For treating acute short-duration pain, the combined agonists only may suffice. However, cases of repeating subacute or chronic persistent pain require inclusion of the low-dose antagonist to maintain analgesia with low doses of both agonists and to avoid development of both mu- and kappa-opioid receptor tolerance and opioid-induced hyperalgesia.

While the most dramatic potentiation of analgesia by applying these methods derives from combining the mu-opioid receptor agonist and ultra-low doses of antagonist, other beneficial interactions of the three combined agents can be achieved. The tolerance and dependence following combined chronic dosing with the kappa-opioid receptor agonist will also be suppressed by including the opioid receptor antagonist, resulting in persistent and prolonged analgesia induced by the kappa-opioid receptor agonist. Interactions between chronic doses of mu-opioid and kappa-opioid receptor agonists further attenuate tolerance and dependence development for both agonist classes.

Activation of excitatory mu-opioid systems by chronic dosing with a mu-opioid agonist involves, at least in part, mobilization of endogenous excitatory amino acid systems (EAA, NMDA, glutamate) as an intermediary. Thus, dextromethorphan and ketamine, NMDA antagonists, reinstate the analgesia of chronic mu-opioid agonists. Less is known about the details of tolerance and dependence of chronically administered kappa-opioid agonists. Gender differences occur in humans to the analgesic efficacy of nalbuphine on repeated dosing after dental surgery (Gear et al., J Pain. April; 9(4):337-41 2008). Females experienced pain relief, while males reacted to the same doses with increased pain. Combining nalbuphine and a sub-analgesic dose of morphine in males reversed the anti-analgesic action of nalbuphine. These authors had previously enhanced and prolonged nalbuphine analgesia by combining the agonist with very low doses of opioid receptor antagonists, the interaction being sensitive to a critical dose ratio.

The order of administration of the three drug components of the invention is not highly critical. It may be simultaneous or separated by time periods short enough to allow for overlapping effects. Referring to routes of administration, oral, intravenous, intramuscular, subcutaneous, sublingual, intrathecal, or transdermal appear acceptable for most of the candidate opioid drugs which would be used.

The compositions, methods, and utility of the invention are directed toward the reduction, amelioration, or suppression of pain in the broadest sense. That is, the intent is to treat all types of pain, including short-term, long term, intermittent or persistent, somatic pain, visceral pain, and neuropathic pain. The invention provides for compositions, uses and methods of administering combined opioid receptor agonists (mu- and kappa1-opioid receptor agonists) and a nonselective opioid antagonist to reduce pain in a subject, wherein the subject may be any mammalian species, including humans.

The invention provides for methods of treating pain in a subject, the method comprising administering to a subject suffering from pain, a moderate dose of a selective mu-opioid receptor agonist, a moderate dose of a selective kappa1-opioid receptor agonist, and an ultra-low dose of a nonselective opioid receptor antagonist, wherein the doses are effective in combination to promote analgesia in the subject and to reduce an adverse side effect of pain treatment with an opioid receptor agonist administered singly in the subject. These methods include administering doses that are effective in combination to provide enhanced analgesia compared to analgesia from a moderate dose of either of said opioid receptor agonists alone.

Treatment of chronic or persistent pain may result in long term administration of an opioid receptor agonist to a subject, and adverse side effects are likely with long term treatment. The invention also provides for methods of enhancing analgesia with an opioid receptor agonist while reducing an adverse side effect of pain treatment with an opioid receptor agonist, the method comprising administering to a subject suffering from pain, a moderate dose of a selective mu-opioid receptor agonist, a moderate dose of a selective kappa1-opioid receptor agonist, and an ultra-low dose of a nonselective opioid receptor antagonist, wherein the doses are effective in combination to promote analgesia in the subject and to reduce an adverse side effect of pain treatment with an opioid receptor agonist in the subject. These methods include administering doses that are effective in combination to provide enhanced analgesia compared to analgesia from a moderate dose of either of said opioid receptor agonist.

The invention also provides for methods of promoting additive analgesia of pain treatment with opioid receptor agonists in a subject while reducing an adverse side effect of pain treatment with an opiate receptor agonist, the method comprising administering to a subject suffering from pain a moderate dose of a selective mu-opioid receptor agonist, a moderate dose of a selective kappa1-opioid receptor agonist, and an ultra-low dose of a nonselective opioid antagonist, wherein the doses are effective in combination to promote analgesia in an additive manner and to reduce an adverse side effect of pain treatment with an opioid receptor agonist in the subject.

The term “selective opioid receptor agonist,” including “selective mu-opioid receptor agonists” and “selective kappa-opioid receptor agonists,” refers to compounds that primarily binds to and activate a specific opioid receptor type.

In any of the methods or uses of the invention, the doses of the three compounds (mu-opioid receptor agonist, kappa-opioid receptor agonist s and opioid receptor antagonist) may be administered as separate compositions simultaneously or within a short time frame before or after the other compositions. Alternatively, the doses of the three compounds may be administered in combination as a single composition. In a further embodiment, the dose of the three compounds are administered as two compositions in which one compositions comprises two of the compounds and a second composition comprises one of the compounds. For example, the mu-opioid receptor agonist and the kappa-opioid receptor agonist are administered as a single composition and the nonselective opioid receptor antagonist is administered as a separate composition simultaneously or within a short time frame before or after the opioid receptor agonists. In some variations of the method or uses of the invention, the doses of a selective mu-opioid receptor agonist and a selective kappa1-opioid receptor agonist are administered in one composition. In addition, in some variations of the methods or uses of the invention, the doses of a selective mu-opioid receptor agonist, selective kappa1-opioid receptor agonist and nonselective opioid receptor antagonist are administered as a single composition.

The separate compositions may be administered as separate compositions simultaneously or within a short time frame. The separate compositions may be administered consecutively within a short time frame in any order. “Combination” refers to more than one active compound or active pharmaceutical ingredient, including for example, a combination of two opioid receptor agonists and nonselective opioid receptor antagonist.

The invention also provides for compositions comprising a moderate dose of a selective mu-opioid receptor agonist, a moderate dose of a selective kappa-opioid receptor agonist, e.g. kappa1-opioid receptor agonist, and an ultra-low dose of a nonselective opioid receptor antagonist, wherein the doses in combination are effective to reduce pain and to reduce an adverse side effect of treatment with an opioid receptor agonist in a subject. The compounds of the invention are therapeutically effective to enhance the analgesic effect of the opioid receptor agonists in the subject. The compositions of the invention are also therapeutically effective to reduce or suppress hyperalgesic effects of long-term administration of an opioid receptor agonist in the subject.

The compositions of the invention may be administered as an unit dose comprising a moderate dose of a selective mu-opioid receptor agonist, a moderate dose of a selective kappa-opioid receptor agonist, e.g. kappa1-opioid receptor agonists, and an ultra-low dose of a nonselective opioid receptor antagonist, wherein the doses in combination are effective to reduce pain and to reduce an adverse side effect of treatment with an opioid receptor agonist in a subject.

In another embodiment, the invention provides for use of a moderate dose of a selective mu-opioid receptor agonist, a moderate dose of a selective kappa1-opioid receptor agonist, and an ultra-low dose of a nonselective opioid receptor antagonist for the preparation of a medicament for reducing pain in a subject while reducing an adverse side effect of treatment with an opioid receptor agonist in a patient suffering from pain, wherein the doses in combination are effective to reduce pain and to reduce an adverse side effect of treatment with an opioid receptor agonist in the subject. Use of the medicament of the invention has the added benefits of enhancing the analgesic effects of an opioid receptor agonist, reducing the hyperalgesic effect of treating pain with an opioid receptor agonist and promoting the additive analgesia of pain treatment with an opioid receptor agonist.

The invention also provides for use of a moderate dose of a selective mu-opioid receptor agonist, a moderate dose of a selective kappa1-opioid receptor agonist, and an ultra-low dose of a nonselective opioid receptor antagonist for the preparation of a medicament for treating a subject suffering from pain with an opioid receptors agonist, wherein the doses administered in combination are effective to enhance the analgesic effect of the opioid receptor agonist in the subject and the administered doses are effective to reduce an adverse side effect of treatment with the opioid receptor agonist in the subject.

The invention further provides for use of a moderate dose of a selective mu-opioid receptor agonist, a moderate dose of a selective kappa1-opioid receptor agonist, and an ultra-low dose of a nonselective opioid receptor antagonist for the preparation of a medicament for reducing the hyperalgesic effect of treating a subject suffering from pain with an opioid receptor agonist, wherein the doses administered in combination are effective to reduce the hyperalgesic effect of the opioid receptor agonist in the subject and the administered doses in combination are effective to reduce an adverse side effect of treatment with the opioid receptor agonist in the subject.

The invention also provides for use of a moderate dose of a selective mu-opioid receptor agonist, a moderate dose of a selective kappa1-opioid receptor agonist, and an ultra-low dose of a nonselective opioid receptor antagonist for the preparation of a medicament for promoting additive analgesia of pain treatment in certain types of pain (cutaneous somatic) with opioid receptor agonists in a subject while reducing the adverse side effects of pain treatment with an opioid receptor agonist in said subject, wherein the doses administered in combination are effective to promote analgesia in an additive manner in the subject and the administered doses in combination are effective to reduce an adverse side effect of pain treatment with an opioid receptor agonist.

The compositions and the doses of the opioid receptor agonists and opioid receptor antagonists may be administered orally, intravenously, sublingually, transmucosally (including buccally), intramuscularly, subcutaneously, intratracheally, intrathecally or transdermally.

In the methods, uses and compositions of the invention, the moderate doses of the selective mu-opioid receptor agonist is a median quantity of agonist that effectively reduces, suppresses or alleviates clinical pain in a subject. Furthermore, a moderate dose of the selective kappa-opioid receptor agonist, such as the kappa1-opioid receptor agonist of the arylacetamide type, is a median quantity of agonist which in addition to effectively reducing suppressing or alleviating clinical pain in a subject, provides additive or synergistic analgesia when combined with a mu-opioid receptor agonist and reduction of an adverse side effect induced by the mu-opioid receptor agonist. In the methods, uses and compositions of the invention, the ultra-low dose of a nonselective antagonist of an opioid receptor is the smallest quantity of a drug that is likely to produce an appreciable therapeutic affect, and further suppresses the tolerance and emergence of mu-opioid receptor agonist and kappa-opioid receptor agonist induced hyperalgesia (OIH), which results in an enhanced analgesia from both agonists.

For example, the invention provides for compositions, uses and methods of administering the mu-opioid receptor agonist hydrocodone (VICODIN) at an exemplary analgesic moderate dose of 5-10 mg, administering the mu-opioid receptor agonist of hydromorphone (DILAUDID) at an exemplary analgesic moderate dose of 0.5-1.3 mg, administering the mu-opioid receptor agonist levorphanol (LEVO-DROMORON) at an exemplary analgesic moderate dose of 0.5-2 mg, administering the mu-opioid receptor agonist oxycodone (PERCODON) at an exemplary analgesic moderate dose of 5-10 mg, administering the mu-opioid receptor agonist methadone (DOLOPHINE) at an exemplary analgesic moderate dose of 5-20 mg, administering the mu-opioid receptor agonist fentyanyl (SUBLIMAZE) at an exemplary analgesic moderate dose of 0.07-025 1 mg, administering the mu-opioid receptor agonist oxymorphone at an exemplary analgesic moderate dose of 10-20 mg.

The invention also provides for compositions, uses and methods of administering the kappa1-opioid receptor agonist that is of the arylacetamide type such as spiradoline or enadoline. For example, the invention provides for compositions, uses and method of administering spiradoline at an exemplary moderate dose of 0.14-0.042 mg, and compositions, uses and methods of administering the kappa1-opioid receptor agonist enadoline at an exemplary moderate does of 0.08-0.12 mg.

The invention provides for compositions, uses and methods of administering the nonselective antagonist is naloxone or naltrexone. The invention provides for compositions, uses and methods of administrating the nonselective antagonist nalxone at the exemplary dose of 25-125 ng and uses and methods of administering the nonselective antagonist naltrexone at the exemplary ultra-low dose of 50-250 ng.

In particular, the invention provides for compositions, uses and methods of administering a combination of two opioid receptor agonists in which the selective mu-opioid receptor agonist is fentanyl and the selective kappa1-opioid receptor agonist is spiradoline, a combination in which the selective mu-opioid receptor is oxymorphone and the selective kappa1-opioid receptor agonist is spiradoline, a combination in which the selective mu-opioid receptor agonist is fentanyl and the selective kappa1 agonist is enadoline, and a combination in which the selective mu-opioid receptor agonist is oxymorphone and the selective kappa1 agonist is enadoline.

In addition, the invention provides for compositions, uses and methods of administering a combination in which the selective mu-opioid receptor agonist is fentanyl, the selective kappa1 agonist is spiradoline and the nonselective opioid-receptor antagonist is naloxone, a combination in which the selective mu-opioid receptor agonist is oxymorphone, the selective kappa1 agonist is spiradoline and the nonselective opioid-receptor antagonist is naloxone, a combination in which the selective mu-opioid receptor agonist is fentanyl, the selective kappa1 agonist is enadoline and the nonselective opioid-receptor antagonist is naloxone, a combination in which the selective mu-opioid receptor agonist is oxymorphone, the selective kappa1 agonist is enadoline and the nonselective opioid-receptor antagonist is naloxone, a combination in which the selective mu-opioid receptor agonist is fentanyl, the selective kappa1 agonist is spiradoline and the nonselective opioid-receptor antagonist is naltrexone, a combination in which the selective mu-opioid receptor agonist is oxymorphone, the selective kappa1 agonist is spiradoline and the nonselective opioid-receptor antagonist is naltrexone, a combination in which the selective mu-opioid receptor agonist is fentanyl, the selective kappa1 agonist is enadoline and the nonselective opioid-receptor antagonist is naltrexone, and a combination in which the selective mu-opioid receptor agonist is oxymorphone, the selective kappa1 agonist is enadoline and the nonselective opioid-receptor antagonist is naltrexone.

“Therapeutic effect” or “therapeutically effective” refers to an effect or effectiveness that is desirable and that is an intended effect associated with the administration of an opioid receptor agonist including when the opioid receptor agonist is administered in combination with an opioid antagonist according to the invention. Such therapeutic effects include. e.g., analgesia, pain relief, decrease in pain intensity, euphoria or feeling good, and calming so as to reduce heart rate, blood pressure and/or breathing rate.

The compositions, methods and uses of the invention reduce, treat, lessen, ameliorate or suppress pain. The term “pain” refers to any type of pain, including e.g. long term persistent pain, chronic pain, acute pain, somatic pain, visceral pain, and neuropathic pain. Visceral pain is of special interest since it is involved in severe and chronic cases of pain in which opioid receptor agonist monotherapy has insufficiencies. Moreover, mu-opioid and kappa-opioid receptor agonists in combination afford synergist and prolonged analgesia against this type of pain, and addition of a nonselective opioid-receptor antagonist, further attenuates tolerance, depression, and emergence of OIH.

The invention provides for compositions, uses and method of administering opioid receptor agonists and antagonist to reduce pain in a subject, wherein the subject may be any mammal including humans, non-human primates, horses and hoofed mammals, canines or felines.

The invention provides for compositions, uses and methods that reduce the adverse side effects resulting from treating a subject with a opioid receptor agonist. The invention also provides for compositions, uses and methods for suppressing the emergence of adverse side effects or eliminating adverse side effects resulting from treating a subject with an opioid receptor agonist. An effective reduction or suppressing of these side effects is determined by comparing the effect resulting from treatment with a single opioid receptor agonist with the effect resulting from administering a combination of a mu-opioid receptor agonist, a kappa-receptor agonist and a opioid receptor antagonist.

“Adverse side effect” refers to a medically undesired consequences other than the one for which a compound or treatment is intended, such as the negative effects produced by a drug, especially on a tissue or organ system other than the consequence sought to be benefited by its administration. The invention provides for uses and methods that reduce the adverse side effects of a mu-opioid receptor agonists. Some exemplary adverse side effects of administration of an opioid-receptor agonist include tolerance, dependence and hyperalgesia, euphoria, anuria, pruritus, allodynia, and seizures. In addition, the invention provides for uses and methods that reduce the opposing adverse side effects such as dysphoria, diuresis, antipruritus, and anti-allodynic induced by kappa-opioid receptors.

“Analgesia” refers to the attenuation, reduction or absence of sensibility to pain, including the provision of pain relief, the enhancement of pain relief, or the attenuation of pain intensity.

The term “analgesic dose” refers to a dose of a composition or drug that effectively reduces, attenuates, eases, suppresses or alleviates pain. An analgesic dose also refers to an amount that results in analgesic efficacy, for example, as measured by a subject with a pain relief score or a pain intensity difference score, at a given time point, or over time, or as compared to a baseline, and includes calculations based on area under the curve such as those plotting Total Pain Relief Score (TOTPAR) or the Sum of Pain Intensity Difference (SPID).

An “analgesic dose of an opioid receptor agonist” refers to an amount of the opioid receptor agonist that causes analgesia in a subject administered the opioid receptor agonist alone, and includes standard doses of the agonist which are typically administered to cause analgesia (e.g., mg doses).

A “hypo-analgesic” amount is a less-than-analgesic amount, including an amount which is not analgesic or is weakly analgesic in a subject administered the opioid receptor agonist alone, and further includes an “anti-analgesic” dosing schedule, which results in an increase in pain. The optimum amounts, for example, of the opioid receptor agonists and the opioid receptor antagonist administered in combination, will of course depend upon the particular agonist and antagonist used, the carrier chosen, the route of administration, and/or the pharmacokinetic properties of the subject being treated, as well as the desired gender-related effects according to the teachings of the present invention.

The foregoing summary is not intended to define every aspect of the invention, and additional aspects are described in other sections, such as the Detailed Description. The entire document is intended to be related as a unified disclosure, and it should be understood that all combinations of features described herein are contemplated, even if the combination of features are not found together in the same sentence, or paragraph, or section of this document.

In addition to the foregoing, the invention includes, as an additional aspect, all embodiments of the invention narrower in scope in any way than the variations specifically mentioned above. With respect to aspects of the invention described as a genus, all individual species are individually considered separate aspects of the invention. With respect to aspects described as a range, all subranges and individual values are specifically contemplated.

Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention. Additional features and variations of the invention will be apparent to those skilled in the art from the entirety of this application, and all such features are intended as aspects of the invention.

### BRIEF DESCRIPTION OF DRAWINGS

FIG. 1 depicts the colorectal distension (CRD) model. The mean Maximum Percentage Analgesic Effect (M.P.E) (±S.E.M.) is plotted. Panel A depicts fentanyl, log doses, at 15 minutes post-injection (peak effect). ED₅₀ (M.P.E.=50%)=0.009 mg/kg (range: 0.06-0.016 mg/kg); n=3-16 per dose. Panel B depicts spiradoline at 15 minutes post injection (peak effect). ED₅₀=0.56 mg/kg (0.25-1.26 mg/kg); n=3-12 per dose. Panel C depicts enadoline at 30 minutes post-injection (peak effect). ED₅₀=0.077 mg/kg (0.04-0.2 mg/kg); n=4-7 per dose. Panel D depicts oxymorphone at 30 minutes post-injection (peak effect). ED₅₀=0.078 mg/kg (0.02-0.126 mg/kg); n=5-9 per dose. Enadoline (kappa-opioid receptor agonist) and oxymorphone (mu-opioid receptor agonist) served as typical agonist-class reference standards.

FIG. 2 depicts the antinociceptive responses in the CRD model. The mean M.P.E. (±S.E.M.) for actual combined doses of opioid agonist pairs (filled circles) vs. additive theoretical plots of single doses for each pair (filled squares) is plotted at 15 minutes post-injections. FE displays fentanyl+enadoline plots, FS fentanyl+spiradoline plots, OE oxymorphone+enadoline plots, and OS oxymorphone+spiradoline plots; n=6-9 per dose. One dose, 0.6 in the OS panel, was significantly different from additive response, p<0.05.

FIG. 3 depicts the antinociceptive responses in the CRD model. The mean M.P.E. (±S.E.M.) of single opioid agonists (fentanyl, spiradoline, enadoline) are compared with combined agonist pairs at two dose levels tested at 15 minutes (Panel A) and 30 minutes (Panel B) post-injection. * denotes additive interactions; ** denotes supra-additive (synergistic) interactions, p<0.05; n=8-10 per dose.

FIG. 4 depicts the antinociceptive responses for combinations of a mu- and kappa-opioid receptor agonists with pretreatment with an opioid receptor antagonist in the CRD model. The mean M.P.E. (±S.E.M.) for agonist-antagonist interactions are displayed for single and combined agonists (fentanyl alone 0.12 mg, spiradoline alone 0.3 mg and a combination of both agonists with pretreatment with an opioid antagonist (beta-funeltrexamine (β-FNA) or norbinaltorphimine (nor-BNI) at 15 minutes (Panel A) and 30 minutes (Panel B) post-injections. Panel A: * denotes a significant increase in M.P.E. for the combination of fentanyl and spiradoline compared to either agonist alone (p<0.01). # denotes a significant reduction in M.P.E. for fentanyl after pretreatment with an opioid receptor antagonist compared to saline pretreatment. @ denotes a significant reduction in M.P.E. for the combination of agonists after pretreatment with an opioid receptor antagonist compared to saline pretreatment (p<0.01). Panel B: ** denotes significant increase in M.P.E. for the combination of fentanyl and spiradoline compared to either agonist alone (p<0.05).

### DETAILED DESCRIPTION

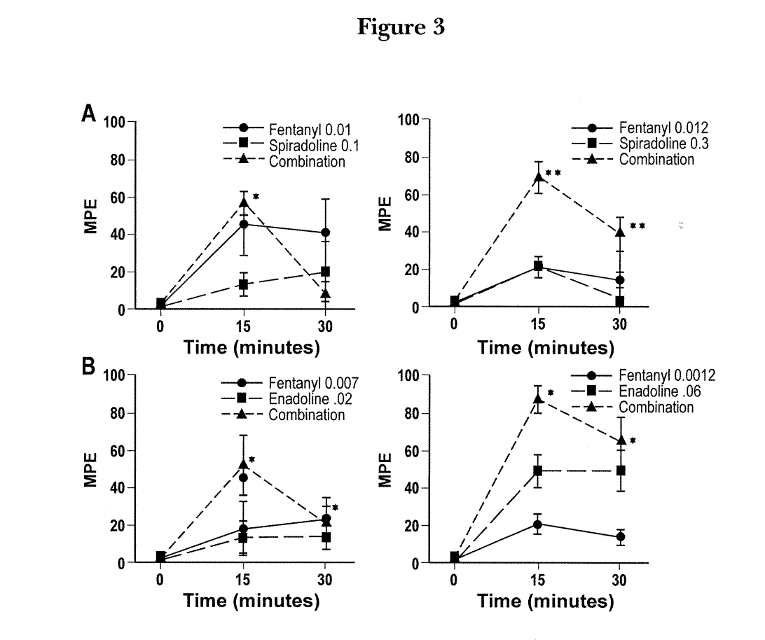

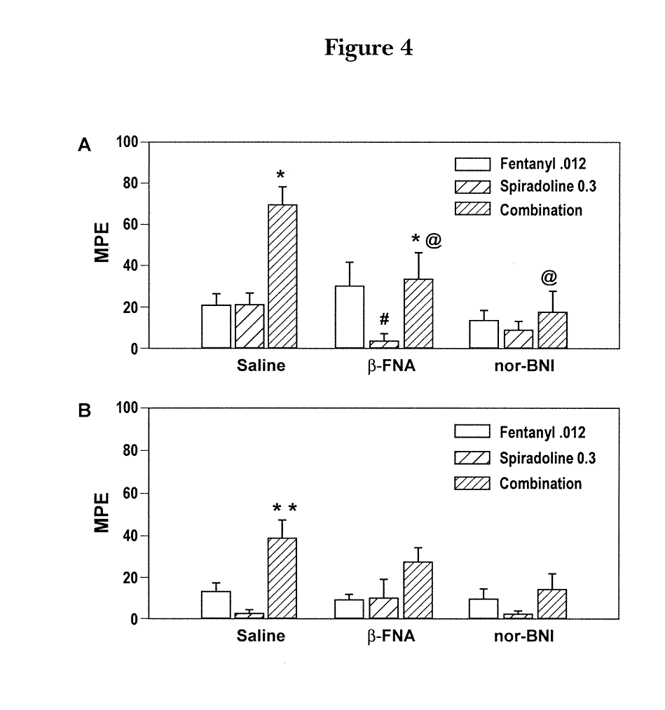

The data provided herein demonstrates that dose-response patterns of individual doses of the opioid receptor agonists, fentanyl and spiradoline, included full antinociceptive effects (ANC) in the CRD visceral pain assay. Comparison of the theoretical combination effects of opioid receptor agonists, measured as added sums of individual opioid receptor agonist responses, with actual combined effects of fentanyl plus spiradoline, indicated primarily additive-response patterns of ANC for these combinations. Higher-dose combinations of fentanyl plus spiradoline produced supra-additive ANC at both 15 and 30 minutes post-injection (FIGS. 3 and 4). Thus, combination of this mu-opioid agonist and this kappa-opioid agonist at some dose levels enhanced the increase in the threshold for visceral pain in the CRD test model beyond an additive level.

Generally, mu- and kappa-opioid agonists induce responses on different receptors and types of pain, and with different efficacies when they both affect the same type of pain or pain test model (Ness et al., Pain. 41: 167-234 (1990)). Spinal ANC was proposed to involve mu2-opioid receptors, while supra-spinal (brain) ANC involved mu1-opioid receptors (Pasternak et al., Life Sci. 138: 1889-98 (1986). Mu-opioid receptors predominate on C fibers, whereas kappa-opioid receptors predominate on A-delta fibers (Werz et al., J. Pharmacol. Exp. Ther. 243: 258-63 (1987)). Visceral pain systems distribute widely, branching and diverging extensively (Bonica, The Management of Pain. 28-94 (1990)), explaining the phenomenon of referred pain. Visceral pain afferents make up only 2-15% of all afferent fibers at various spinal cord levels (Ness et al., Pain. 41: 167-234 (1990)). The ratio of A-delta/C fibers in primary visceral afferents is ⅛- 1/10 (promoting diffuse, widespread activity), while the ratio in dorsal root is 2/1 (favoring focal, discriminated reactions) (Janig et al., Prog. Brain Res. 67: 87-114 (1986)).

Studies, such as those described in Miaskowski et al. (Brain Res. 608: 87-94 (1993)) demonstrate that mu- and kappa-opioid agonist interactions produce antagonistic or synergistic ANC, the latter accompanied by reduced side effects. These studies used a mechanical nociceptive stimulus, which implies visceral-type pain mechanisms. Intracerebroventricular (i.c.v.) injections delivered agonists to brain sites while intrathecal (i.t.) injections delivered them to spinal sites. Antagonism was seen with i.c.v. injections of DPDPE (delta-opioid receptor agonist) combined with i.t. injections of DAMGO (mu-opioid receptor agonist). However, most combinations produced enhanced ANC, the greatest synergy seen after combined i.c.v. injections of DAMGO and i.t. injections of U50,488H.

Results of these studies led to the proposed mechanism of multiple brain-spinal ascending-descending neuronal loops, with mu- and kappa-opioid receptors residing at junctions of shared components. Multiple agonist actions at receptors in serial or parallel arrangements were considered to amplify the total ANC effect beyond the sum of the parts.

Consistent with the above theories, supra-spinal dynorphin (endogenous kappa-opioid receptor agonist) antagonized the ANC of morphine also injected supra-spinally, but supra-spinal dynorphin potentiated spinally-induced morphine ANC (Ren et al., Peptides. 6: 1015-20 (1985)). Also, Stachura et al. (Pol. J. Pharmacol. 45: 37-41 (1994)) reported potentiated ANC of SC morphine by spiradoline injected intrathecally. ANC from morphine or U50, 488H in mice was attenuated by increasing brain GABA activity or reducing brain 5HT activity (Nemmani et al., Neuropharmacology. 44: 304-10 (2003)), indicating that complex multiple interactions between opioid receptor agonists and other neurotransmitter systems also occur.

Neurochemical studies support these hypotheses. Both mu- and kappa-opioid receptors were found on most nociceptive neurons throughout central and peripheral mammalian nervous systems (Atweh et al, Brain Res. 124: 53-67 (1977); Allerton et al., Brain Res. 502: 149-57 (1989)). Interactions may occur on peripheral A-delta fibers and C fibers, on dorsal root ganglion cells and synaptic endings, and on interneurons in dorsal horn or spinal projection cells. Also, interactions occur in supra-spinal nuclei (especially PAG, PVG, RVM, and raphe nuclei), as well as in forebrain loci (see Bie et al., J. Neurosci. 32: 7262-8 (2003), and He et al., J. Pharmacol. Exp. Ther. 280: 1210-4 (1997)).

In addition, kappa-opioid receptors were found to be involved in the same neuronal network in rat PAG that controls morphine tolerance and dependence (Herra'ez-Baranda et al. Brain Res. 137:166-73, (2005)). This discovery relates to studies by He et al., J. Pharmacol. Exp. Ther. 280: 1210-4 (1997), Jang et al., Arch. Pharm. Res. 29: 677-84 (2006), Song et al., Life Sci. 51: 107-11 (1992), Tao et al., Eur J Pharmacol. 1994 May 2;256(3):281-6.Links (1994), and Yamamoto et al., Eur. J. Pharmacol. 156: 173-6 (1988), in which kappa-opioid agonists enhanced morphine ANC, reversing tolerance and/or dependence. Acute mu- and kappa-opioid agonists both inhibited glutamate input to brain-stem ventral tegmental area neurons, but from different sources (Margolis et al., J. Neurophysiol. 93: 3086-93 (2005)). But chronic opioid agonists activate glutamate mechanisms, promoting opioid-agonist tolerance and dependence (Fundytus, CNS Drugs. 15: 29-85 (2001)). These types of neuronal dichotomy would allow for potential synergistic or occlusive effects of combined agonists. Complex mu-/kappa-opioid interactions, with differential relationships of opioid receptors in visceral and cutaneous types of pain, were also elaborated by Schmauss et al. J. Pharmacol. Exp. Ther. 228: 1-12 (1984) and Gebhart, Animal Pain. 81-93 (1992)).

The majority opinion of pain therapists today still adhere to the use of mu-opioid receptor agonist monotherapy as the appropriate treatment for subacute or chronic moderate to severe pain. These therapists remain convinced that kappa-opioid receptor agonists have only a minor role in pain management. Still, some authorities have recently promoted the use of combined opioids or opioid/non-opioid analgesics to overcome the problems of mu-opioid receptor agonist monotherapy (Coop et al., Amer. J. Pharm. Educ. 24: 198-205 (2002); McNaull et al., Eur J Pharmacol. 560(2-3):132-41 (2007); Tucker et al., BMC Anesthesiol. 5(1):2. (2005); Olmstead et al., Psychopharmacology (Berl). 181(3):576-81. (2005)). However, much of the opioid scientific community continues to conclude that all kappa-opioid receptor agonists are antagonists of mu-opioid receptor agonist analgesia. For example, McNally et al. (Neuroscience. 112(3):605-17 (2002)), cite in their review article the work of Pan and colleagues in order to emphasize the incompatibility of mu-kappa-opioid combinations in pain therapy and to demonstrate that the opioid scientific community had established the fact that kappa-opioid receptor agonists interfered with the analgesic effects of mu-opioid receptor agonists.

Currently, drug development comprising analgesic combinations of mu-opioid receptor agonists and kappa1-opioid receptor agonists to manage subacute or chronic severe pain has been slow. This disinterest is likely fostered by, at least partly, the complexities and difficulties of analyzing combination-drug studies and their potential to increase research expense and delay or hamper approval of candidate drugs for clinical use. In addition, there is an ingrained prejudice in the field against drug-combination therapy. Nevertheless, some drug industry scientists are exploring opioid and nonopioid combinations to potentiate analgesia while reducing adverse side effects (see Baker et al., Pharmacol Biochem Behav. 74(1):73-86 (2002)).

Ultra-low doses of opioid antagonists enhance opioid-induced analgesia and attenuate the adverse side effects of tolerance and withdrawal. For example, small doses of naltrexone (antagonist) were co-administered with mu-opioid receptor agonists, such as morphine or oxycodone, to test for altered reward effects or aversive effects of precipitated withdrawal (Olmstead et al., Psychopharmacology (Berl). 81(3):576-81 (2005)). In these studies, pretreatment with naltrexone blocked the conditioned place preference (CPP) of morphine and co-administration of naltrexone blocked the conditioned place aversion (CPA) to withdrawal from chronic oxycodone. It was proposed that the effect of naltrexone on CPP training with oxycodone yielded a bi-phasic dose-pattern effect, the middle natrexone dose lacked an effect, and high naltrexone doses blocked CPP. Thus, it was concluded that ultra-low doses of natrexone interfere with the rewarding effects of analgesic doses of mu-opioid receptor agonists, in addition to suppressing their aversive withdrawal effects.

The present invention provides for compositions and methods of reducing or treating pain with a combination of a mu-opioid receptor agonist, a kappa-opioid receptor agonist, and a nonselective opioid antagonist. These methods are directed to reducing and treating any type of pain, including for example long term persistent pain, chronic pain, subacute pain and acute pain. Chronic pain is continuous or recurrent. Acute pain occurs in brief periods of time and is associated with temporary disorders. The pain may be slight, moderate or severe. The types of pain that may be reduced or treated with the methods and compositions of the invention including nociceptive pain such as somatic pain and visceral pain, neuropathic pain, muscle pain, colicky pain, and referred pain. The invention provides for method of reducing or treating pain caused by any source including postoperative pain and pain associated with chronic diseases.

The term “opioid” refers to “opioid-like compounds” or compounds or compositions including substituents of such compounds or compositions which bind to specific opioid receptors and have agonist (binding/activation) or antagonist (inactivation) effects at these receptors, such as opioid alkaloids, including the agonist morphine and its metabolite morphine-6-glucuronide and the antagonist naltrexone and its metabolite and opioid peptides, including enkephalins, dynorphins and endorphins. The opioid can be obtained from an opiate base or can be an opioid synthesized pharmaceutically as an acceptable salt. The pharmaceutically acceptable salt may be an inorganic or an organic salt. Representative salts include hydrobromide, hydrochloride, mucate, succinate, n-oxide, sulfate, malonate, acetate, phosphate dibasic, phosphate monobasic, acetate trihydrate, bi(heplafluorobutyrate), maleate, bi(methylcarbamate), bi(pentafluoropropionate), mesylate, bi(pyridine-3-carboxylate), bi(trifluoroacetate), bitartrate, chlorhydrate, fumarate and sulfate pentahydrate. The term “opiate” refers to drugs derived from opium plants.

An “opioid receptor agonist” or “opioid agonist” is an opioid compound or composition including any active metabolite of such compound or composition that binds to and/or activates opioid receptors, for example, binds to those receptors on nociceptive neurons which mediate pain such as binding and/or activating mu- or kappa-opioid receptors. Such agonists have analgesic activity (with measurable onset, peak, duration and/or total effect) and can produce analgesia. Opioid agonists include: alfentanil, allylprodine, alphaprodine, anileridine, apomorphine, apocodeine, benzylmorphine, bezitramide, buprenorphine, butorphanol, clonitazene, codeine, cyclazocine, cyclorphen, cyprenorphine, desomorphine, dextromoramide, dezocine, diampromide, dihydrocodeine, dihydromorphine, dimenoxadol, dimepheptanol, dimethylthiambutene, dioxyaphetyl butyrate, dipipanone, eptazocine, ethoheptazine, ethylmethylthiambutene, ethylmorphine, etonitazene, fentanyl, heroin, hydrocodone, hydroxymethylmorphinan, hydromorphone, hydroxypethidine, isomethadone, ketobemidone, levallorphan, levorphanol, levophenacylmorphan, lofentanil, meperidine, meptazinol, metazocine, methadone, methylmorphine, metopon, morphine, myrophine, nalbuphine, narceine, nicomorphine, norlevorphanol, normethadone, nalorphine, normorphine, norpipanone, ohmefentanyl, opium, oxycodone, oxymorphone, papaveretum, pentazocine, phenadoxone, phenomorphan, phenazocine, phenoperidine, pholcodine, piminodine, piritramide, propheptazine, promedol, profadol, properidine, propiram, propoxyphene, remifentanil, sufentanil, tramadol, tilidine, salts thereof, mixtures of any of the foregoing. Opioid receptor agonists include naturally occurring opiates and pharmaceutically synthetic opioids.

“Bimodally-acting opioid agonists” are opioid agonists that bind to and activate both inhibitory and excitatory opioid receptors on nociceptive neurons which mediate pain. Activation of inhibitory receptors by said agonists causes analgesia. Activation of excitatory receptors by said agonists results in anti-analgesia, hyperexcitability, hyperalgesia, as well as development of physical dependence, tolerance and other undesirable side effects. Bimodally-acting opioid agonists may be identified by measuring the opioid's effect on the action potential duration (APD) of dorsal root ganglion (DRG) neurons in tissue cultures. In this regard, bimodally-acting opioid agonists are compounds which elicit prolongation of the APD of DRG neurons at pM-nM concentrations (i.e., excitatory effects), and shortening of the APD of DRG neurons at μM concentrations (i.e., inhibitory effects).

An “opioid antagonist” is an compound or composition including any active metabolite of such compound or composition that in a sufficient amount attenuates (e.g., blocks, inhibits, or competes with) the action of an opioid agonist. An “effective antagonistic amount” is one which attenuates the analgesic effectively activity of an opioid agonist. An opioid antagonist binds to and blocks (e.g., inhibits) opioid receptors, for example, binds to and blocks receptors on nociceptive neurons which mediate pain. Opioid antagonists according to the present invention include: naltrexone, naloxone nalmefene, naloxone methiodide, nalorphine, naloxonazine, nalide, nalmexone, nalbuphine, nalorphine dinicotinate, naltrindole (NTT), naltrindole isothiocyanate, (NTII), naltriben (NTB), nor-binaltorphimine (nor-BNI), b-funaltrexamine (β-FNA), BNTX, cyprodime, ICI-174,864, LY117413, MR2266, or an opioid antagonist having the same pentacyclic nucleus as nalmefene, naltrexone, nalorphine, nalbuphine, thebaine, levallorphan, oxymorphone, butorphanol, buprenorphine, levorphanol, meptazinol, pentazocine, dezocine, or their pharmacologically effective esters or salts. An opioid antagonist with partial agonist activity is cholera toxin B.

A “mixed opioid receptor agonist/antagonist is a compound that has an affinity for two or more types of opioid receptors and blocks opioid effects on one receptor type while producing opioid effects on a second receptor type. Mixed opioid receptor agonist/antagonists include compounds that exhibit opioid receptor agonist action at one dose and have an antagonistic action at another dose.

The term “moderate analgesic dose” refers to a median quantity of a drug that effectively reduces, suppresses or alleviates pain suffered by a subject. An exemplary moderate dose is the ED₅₀ as this dose exerts an effective therapeutic action that is not near-maximal dose. A near maximal effect is exerted at the ED-80 or ED-90 dose level.

The invention also provides for compositions, uses and methods of administering the kappa1-opioid receptor agonist spiradoline at an exemplary moderate dose of 0.14-0.42 mg, and administering the kappa1-opioid receptor agonist enadoline at an exemplary moderate does of 0.08-0.12 mg.

The term “ultra-low dose” refers to a very small quantity relative to the well-established/conventional larger quantities that are known to produce an appreciable therapeutic effect. Generally, it can be expected that an ultra-low dose will produce a different effect than the well-established higher dose. An ultra-low dose of nonselective antagonist is a quantity that is effective for antagonizing mu- and kappa-excitatory opioid receptors but that is below the threshold for antagonizing mu- and kappa-inhibitory opioid receptors. An exemplary threshold for antagonizing mu- and kappa-inhibitory opioid receptors is 5 mg of naltrexone.

### Adverse Side Effects

An “adverse side effect” of an opioid receptor agonist is a medically undesirable significant consequence of administration and this consequence is an effect other than the effect for which the opioid receptor agonist is intended. For examples, an adverse side effect of an opioid receptor agonist is a consequence other than amelioration or reduction or suppression or attenuation of pain. Exemplary adverse side effects of administration of opioid receptor agonists including hyperalgesia, tolerance, nausea, vomiting, dizziness, somnolence/sedation, pruritus allodynia, reduced gastrointestinal motility including constipation, peripheral vasodilatation including leading to orthostatic hypotension, headache, dry mouth, sweating, asthenia, dependence, mood changes (e.g., dysphoria, euphoria), mental clouding, lethargy, impairment of mental and physical performance, anxiety, fear, depression of the cough reflex; miosis, clouded sensorium, skin rash, release of histamine, lightheadedness, ureteral spasm; spasm of vesical sphincter and urinary retention; and tramadol: seizures; anaphylactoid reactions (lessened resistance to toxins), diarrhea; anuria, CNS stimulation (“CNS stimulation” is a composite that can include nervousness, anxiety, agitation, tremor, spasticity, euphoria, emotional lability and hallucinations); malaise, confusion, coordination disturbance, euphoria, nervousness, sleep disorder; abdominal pain, anorexia, flatulence, hypertonia, rash, visual disturbance, menopausal symptoms, urinary frequency, and urinary retention. An adverse side effect may be a serious adverse side effect such as respiratory depression or also apnea, respiratory arrest, circulatory depression, cardiac arrest, hypotension or shock.

### Mu-Opioid Receptor Agonists

A “mu-opioid receptor agonist” is an opioid receptor agonist that primarily binds to and/or interacts with mu-opioid receptors and from such interactions produces its therapeutic effects (e.g., analgesic activity). Excluded from the definition of mu-opioid receptor agonists are agents that primarily bind to and activate kappa-opioid receptors, and from such interactions produce their therapeutic effects (e.g., analgesic activity).

Exemplary mu-opioid receptor agonist include hydrocodone (VICODIN), hydromorphone (DILAUDID), levorphanol (LEVO-DROMORON), oxycodone (PERCODON), methadone (DOLOPHINE), fentyanyl (SUBLIMAZE), sufentanil and morphine. Adverse side effects that are particularly observed with monotreatment of a mu-opioid-receptor agonist are euphoria, anuria, pruritus allodynia, and seizures.

The invention provides for compositions comprising a moderate analgesic dose of a mu-opioid receptor agonist and methods of administering a moderate analgesic dose of a mu-opioid-receptor agonist. Exemplary moderate analgesic dose ranges of hydrocodone (VICODIN) include 1-25 mg, 1-10 mg, 2-12 mg, 5-10 mg, 5-15 mg, 10-20 mg, and 15-25 mg. Exemplary moderate analgesic dose ranges of hydromorphone (DILAUDID) include 0.25-5.0 mg, 0.25-1.5 mg, 0.5-2 mg, 0.5-1.0 mg, 0.6-1.2 mg, 0.75-1.25 mg, 0.8-1.3 mg, 0.9-1.5 mg, 1.0-2.0 mg, 1.0-2.5 mg, and 2.5-5.0 mg. Exemplary moderate analgesic dose ranges include levorphanol (LEVO-DROMORON) is 0.25-5.0 mg, 0.25-5.0 mg, 0.5-1.0 mg, 0.5-2.0 mg, 0.6 mg -1.2 mg, 0.75-1.25 mg, 0.8-1.3 mg, 0.9-1.5 mg, 1.0-2.0 mg, 1.0-2.5 mg, and 2.5-5.0 mg. Exemplary moderate analgesic dose ranges of oxycodone (PERCODON) include 1-25 mg, 1-20 mg, 2.5-25 mg, 2.5-10 mg, 5-10 mg, 5-20 mg, 7.5-20 mg, 10-20 mg, and 15-25 mg. Exemplary moderate analgesic dose ranges of methadone (DOLOPHINE) include 1-25 mg, 1-20 mg, 2.5-25 mg, 2.5-10 mg, 5-10 mg, 5-20 mg, 7.5-20 mg, 10-20 mg, and 15-25 mg. Exemplary moderate analgesic dose ranges of mu-opioid receptor agonist fentyanyl (SUBLIMAZE) include 0.01-0.5 mg, 0.01-0.5 mg, 0.05-0.1 mg, 0.07-0.25 mg, 0.08-0.3 mg, 0.09-0.4 mg and 0.1-0.5 mg. Exemplary moderate analgesic dose ranges of mu-opioid receptor agonist oxymorphone (OPANA) include 1-25 mg, 1-20 mg, 2.5-25 mg, 2.5-10 mg, 5-10 mg, 5-20 mg, 7.5-20 mg, 10-20 mg, and 15-25 mg.

### Kappa-Opioid Receptor Agonists

A “kappa-opioid receptor agonist” is an opioid agonist that primarily binds to and/or activates kappa-opioid receptors and from such interactions produces its therapeutic effects (e.g., analgesic activity), including, for example, pentazocine, nalbuphine and butorphanol. Excluded from the definition of kappa-opioid-receptor agonists are opioid receptor agonists that primarily bind to or activates mu opioid-receptor agonists. Kappa-opioid receptor agonists induce antinociceptive effects. Mixed partial agonists act at mu- and kappa-opioid receptors, such as butorphenol and nalbuphine are also considered “kappa-opioid receptor agonists.”

Exemplary kappa-opioid receptor agonists include arylacetamide kappa agonists including spiradoline, enadoline, U50,488, pentazocine, dinorphin, bremozocine, PD117302, U69593, MR2034, cyclazocine, ethylketocyclazonine, ketazocine, butorphanol and nalbuphine. Adverse side effects that are particularly observed with monotreatment of a kappa-opioid receptor agonist are dysphoria, diuresis, antipruritus, anticonvulsant and anti-allodynic.

Additional exemplary kappa-opioid receptor agonists include those described by: U.S. Pat. No. 4,923,863 hereby incorporated by reference in its entirety (e.g., morpholine derivatives); U.S. Pat. No. 6,110,947 hereby incorporated by reference in its entirety (e.g., pyrrolidinyl hydroxamic acid compounds); U.S. Pat. No. 5,965,701 hereby incorporated by reference in its entirety (e.g., kappa-opioid receptor peptides with affinity for the kappa-opioid receptor at least 1,000 times greater than its affinity for the mu-opioid receptor).

Butorphanol, is a mixed partial agonist at mu- and kappa-opioid receptors is considered one of the most potent agonists (effective at a human dose of 2 mg). Nalbuphine, is a less potent mixed partial opioid that is an agonist at kappa-opioid receptors and an antagonist at mu-opioid receptors (effective at a human dose of 5-10 mg). Pentazocine is also a mixed partial agonist at mu- and kappa-opioid receptors (Hardman et al., Goodman and Gilman's The Pharmacological Basis of Therapeutics. 81-93 (1996); Walker et al., Psychopharmacology. 155: 362-71 (2001). Arylacetamide kappa-opioid agonists (U50,488H, spiradoline, and enadoline) are selective, without direct mu-opioid effects (von Voigtlander and Lewis, 1988). Antinociception of U50,488H involves 5HT, being attenuated by 5HT antagonists, and GABA disinhibitory effects, but spiradoline antinociception is less dependent upon serotonin interactions.

The invention provides for compositions comprising a moderate analgesic dose of a mu-opioid receptor agonist and methods of administering a moderate analgesic dose. Exemplary moderate analgesic dose ranges of spiradoline includes 0.2-0.50 mg, 0.2-0.45 mg, 0.015-0.45 mg, 0.12-0.42 mg, and 0.1-0.4 mg. Exemplary moderate analgesic dose ranges of enadoline include 0.1-0.25 mg, 0.1-0.20 mg, 0.08-0.12 mg, 0.08-0.15 mg, 0.05-0.12 mg, and 0.05-0.10 mg.

### Nonselective Antagonists

A “nonselective opioid antagonist” is an opioid receptor antagonist that binds to/or interacts with more than one opioid receptor and primarily blocks or suppresses agonist binding to or activation of at least two opioid receptors in the presence of an agonist. The nonselective opioid antagonist will block the mu-opioid receptors, kappa-opioid receptors and/or the delta-opioid receptors and thereby block or inhibit the opioid receptor's therapeutic effects (e.g., analgesic activity). Exemplary nonselective opioid receptor antagonists include naloxone (NLX), naltrexone (NTX), nalmefene, and diprenorphine.

The invention provides for compositions comprising an ultra-low dose of a nonselective opioid receptor antagonist and methods of administering an ultra-lose dose of a nonselective opioid receptor antagonist. Exemplary ultra-low dose ranges of naltrexone include 10 ng-500 ng, 50-250 ng, 60-200 ng, 75 ng-100 ng and 50-100 ng. Exemplary ultra-low doses of naloxone include 10-250 ng, 15-200 ng, 25-125 ng, 30 ng-100 ng and 50 ng-100 ng.

### Animal Models

The effectiveness of the claimed methods and analgesic compositions may be demonstrated in human clinical trials and in animal models. Exemplary animal models include those in which the nociceptive stimulus is mechanical, electrical, thermal or chemical.

One exemplary animal model is the colorectal distension (CRD) assay which serves well as a visceral pain test model in cats, dogs and rats (Briggs et al. Pharmacol Biochem Behav 60: 467-72, 1998). In this assay, pressurized air pulse-stimuli delivered to a balloon rectal catheter are used as a nociceptive stimulus in a restrained animal. The pressure-pulse may be gradually increased and the nociceptive threshold is measured by abdominal contraction.

Nociceptive thresholds may be established in the colorectal distension assay (CRD) in restrained subjects by air-pressure pulse-stimuli, inflating the balloon-catheter. To insure a standard, reproducible, brief stimulus, a stimulus-pulse shaper may be used. The nociceptive stimulus is delivered by opening the line from pressurized reservoir to the catheter (placed within the subject's rectum), then from catheter to the open air over a maximal period of one second. Thus, at least 6 stimuli could be delivered over the span of one minute. Two stimuli are delivered within 10 seconds, yielding essentially identical signals to establish a valid response. The air pressure to the balloon catheter placed in the rectum activates noriceptives in the intestine wall to induce a “guarding response.” Another example of an animal model is the rat tail flick test as described by D'Amour et al., J Pharmacol Exp Ther. 1941;72:74-9 (1941). In this test, the tail of a trained restrained rat is dipped into a cold solution, such as a solution of ethylene glycol and water maintained at −10° C., or a hot solution, such as water bath at 52° C., or exposed to a radiant heat, as a nociceptive stimulus. The nociceptive threshold is determined by establishing latency from the time the tail is dipped until the time the rat flicked its tail from the cold solution as described in.

A mechanical stimulus, such as pressure, may be used as a nociceptive stimulus. Pressure in these tests may be applied progressively or by gradual increases. The pressure is administered by a method that allows for measurable increments. The nociceptive threshold may be measured by the length in time or the amount of pressure applied before the animal withdraws the paw or tail, the animal tries to release its trapped limb or in a vocalization, such as the Randall-Selitto assay as described by Anseloni et al., J, Neurosci. Methods 131 (1-2): 93-97, 2003).

In the paw withdrawal test as described by Hargreaves et al., Pain. January;32(1):77-88.(1988), a nociceptive stimulus, such as radiant heat, is applied to the paw that has been inflamed by an agent, such as subcutaneous injection of carrageenin or exposure to UV light. The nociceptive threshold is determined by the length of time between exposure to the nociceptive stimulus and withdrawal of the paw from that stimulus.

Another exemplary animal model is the hot plate test, which is carried out by introducing the animal to an open-ended cylindrical space with a metallic floor that is heated to a constant temperature by a thermode or boiling water as described by Woolfe et al., J. Pharmacol. Exp. Ther. 80:300-307. (1944). Reaction time to the heated plate is determined by observing paw licking or jumping.

Electrical stimuli may also be used in animal models of pain. For example, electrical stimulation of the tail may be delivered as long lasting, gradually increasing, intensities through a subcutaneous electrode inserted in the tail of a rat or mouse as described by Carroll et al., Arch. Int. Pharmacodyn. Ther. 125: 383-403 (1960). The nociceptive threshold may be measured by observing successive reflex movement of the tail, vocalization at the time of stimulation and then vocalization continuing beyond the period of stimulation. Alternatively, an electronic stimulus may by delivered through electrically charged cage floors such as described by Blake et al. Med Exp 9: 146-150 (1963). The nociceptive threshold is measured by behaviors such as animal twitching, vocalization or attempting to escape the cage (the flinch-jump test). The electronic stimulus may be administered as single shocks or for very short time periods. As the stimulation increases, the following responses are observed successively: twitching, escape behavior, vocalization and biting the electrodes. These responses are hierarchically organized and the nociceptive threshold may be analyzed by the sensitivity to the test, as described by Nilsen Acta Pharmacol Toxicol (Copenh) 18:10-22. (1961).

For example, treatments that have an analgesic effect will cause the animal to have a higher nociceptive threshold, such as the animal will endure the nociceptive stimuli for a longer time. For the present invention, administration of the combination of a mu- and a kappa-opioid receptor agonist in the rat tail flick test would cause a rat to endure the norciceptive stimuli for a longer time period than if the animal received a comparable dose of a single opioid receptor agonist. Administration of a mu- and a kappa-opioid receptor agonist in combination with an ultra-low dose of a nonselective opioid receptor antagonist will cause the animal to have a lower nociceptive threshold, such as the animal will endure the nociceptive stimuli for a shorter time period compared to those animals are administered the mu- and kappa-opioid receptor agonists in the absence of a nonselective opioid receptor antagonist.

Assays to measure adverse side effects caused by administration of opioid receptor agonists are well known in the art. For example, the acetic acid writhing test in rodents, as described by Litchfield et al., J. Pharmacol. Exp. Ther. 96: 99-113 (1949), may be used to measure the twisting movements or struggling behavior induced by opioid receptor agonist administration. For example, the mu- and kappa-opioid receptor agonists and a nonselective opioid receptor antagonist is administered according to any of the methods of the invention. At various time points after this treatment, acetic acid (e.g. 0.7% acetic acid solution) is injected intraperitoneally (e.g. 30, 60, 120, 180 and 240 min after treatment). Ten minutes after the injection of acetic acid, the writhing responses are counted for a set time period, wherein an increase in the number of twisting movements indicates an adverse side effect.

Any of the above-described assays, such as the cold water tail flick assay and the CRD assay, may be modified to measure an adverse side effect such as tolerance, dependence and pruitius (itching). In addition, scratching responses induced by administration of opioid receptor agonists may be monitored by videotaping the treated animals for a set time period as described in Ko & Naughton, (Anesthesiology 92(3): 795-805, 2000), wherein an increase in scratching indicates an adverse side effect.

### Pharmaceutical Compositions

The composition may be administered to the subject by known procedures including but not limited to oral, sublingual, transmucosal (including buccal), intramuscular, subcutaneous, intravenous, intratracheal, intrathecal or transdermal modes of administration. When a combination of these compounds are administered, they may be administered together in the same composition, or may be administered in separate compositions. If the opioid receptor agonists and the opioid receptor antagonist are administered in separate compositions, they may be administered by similar or different modes of administration, or may be administered simultaneously with one another, or shortly before or after the other.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The opioid agonists and the opioid antagonists may be formulated in compositions with a pharmaceutically acceptable carrier. The carrier must be “acceptable” in the sense of being compatible with the other ingredients of the formulation and not deleterious to the recipient thereof. Examples of suitable pharmaceutical carriers include lactose, sucrose, starch, talc, magnesium stearate, crystalline cellulose, methyl cellulose, carboxymethyl cellulose, glycerin, sodium alginate, gum arabic, powders, saline, water, among others. The formulations may conveniently be presented in unit dosage and may be prepared by methods well-known in the pharmaceutical art, by bringing the active compound into association with a carrier or diluent, as a suspension or solution, or optionally with one or more accessory ingredients, e.g., buffers, flavoring agents, surface active agents, or the like. The choice of carrier will depend upon the route of administration.

The opioid agonists or opioid antagonists may be provided in the form of free bases or pharmaceutically acceptable acid addition salts. As used herein, “pharmaceutically acceptable salts” refer to forms of the disclosed compounds wherein the therapeutic compound is modified by making acid or base salts thereof. The “pharmaceutically acceptable salt” embraces an inorganic or an organic salt.

Examples of pharmaceutically acceptable salts include, but are not limited to, mineral or organic acid salts of the opioid antagonist or opioid agonist. The pharmaceutically acceptable salts include the conventional non-toxic salts made, for example, from non-toxic inorganic or organic acids. For example, such conventional non-toxic salts include those derived from inorganic acids such as hydrochloric, hydrobromic, sulfuric, sulfonic, sulfamic, phosphoric, nitric, and others known to those skilled in the art; and the salts prepared from organic acids such as amino acids, acetic, propionic, succinic, glycolic, stearic, lactic, malic, malonic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, glucuronic, and other acids. Other pharmaceutically acceptable salts and variants include mucates, phosphate(dibasic), phosphate(monobasic), acetate trihydrate, bi(heptaflourobutyrate), bi(methylcarbamate), bi(pentaflouropropionate), mesylate, bi(pyridine-3-carboxylate), bi(triflouroacetate), bitartrate, chlorhydrate, and sulfate pentahydrate. An oxide, though not usually referred to by chemists as a salt, is also a “pharmaceutically acceptable salt” for the present purpose. For acidic compounds, the salt may include an amine-based (primary, secondary, tertiary or quaternary amine) counter ion, an alkali metal cation, or a metal cation. Lists of suitable salts are found in texts such as Remington's Pharmaceutical Sciences, 18^{th} Ed. (Alfonso R. Gennaro, ed.; Mack Publishing Company, Easton, Pa., 1990); Remington: the Science and Practice of Pharmacy 19^{th} Ed. (Lippincott, Williams & Wilkins, 1995); Handbook of Pharmaceutical Excipients, 3.sup.rd Ed. (Arthur H. Kibbe, ed.; Amer. Pharmaceutical Assoc., 1999); the Pharmaceutical Codex: Principles and Practice of Pharmaceutics 12^{th} Ed. (Walter Lund ed.; Pharmaceutical Press, London, 1994); The United States Pharmacopiea: The National Formulary (United States Pharmacopeial Convention); and Goodman and Gilman's: the Pharmacological Basis of Therapeutics (Louis S. Goodman and Lee E. Limbird, eds.; McGraw Hill, 1992), the disclosures of which are hereby incorporated by reference.

“Unit dose form” or “unit dosage form” refers to physically discreet units suitable as unitary doses for human subjects or veterinary subjects, each unit containing a predetermined quantity of active material calculated to produce the desired therapeutic effect (e.g., analgesia), in association with a suitable pharmaceutical carrier. Thus, the active ingredients according to the invention (e.g., opioid receptor agonist, opioid receptor antagonist, or other active pharmaceutical ingredient) either each alone or in combination may conveniently be presented to the subject for administration in unit dose form.

For oral or sublingual administration, including transmucosal, the formulation may be presented as capsules, tablets, caplets, pills, powders, granules or a suspension, prepared by conventional means with pharmaceutically acceptable excipients, e.g., with conventional additives or fillers such as lactose, mannitol, corn starch or potato starch; with binders or binding agents such as crystalline cellulose, cellulose derivatives, acacia, corn starch (including pregelatinized) or gelatins; with disintegrators or disintegrants such as corn starch, potato starch or sodium carboxymethyl-cellulose; or with lubricants or wetting agents such as talc or magnesium stearate. Tablets may be coated, including by methods well known in the art. The formulation may be presented as an immediate-release or as a slow-release, sustained-release or controlled-release form. The formulation may also be presented as a solid drug matrix. Oral dose forms for human administration include: codeine, dihydrocodeine (e.g., SYNALGOS-DC from Wyeth-Ayerst Pharmaceuticals), fentanyl (e.g., ACTIQ from Abbott Laboratories), hydrocodone (e.g., VICODIN and VICOPROFEN from Knoll Laboratories; NORCO from Watson Laboratories; HYCODAN from Endo Pharmaceuticals; NORCET from Abara; ANEXSIA, HYDROCET, and LORCET-HD from Mallinckrodt; LORTABS from UCB Pharna; HY-PHEN from Ascher; CO-GESIC from Schwarz Pharma; ALLAY from Zenith Goldline), hydromorphone (e.g., DILAUDID from Knoll), levorphanol (e.g., LEVO-DROMORAN from ICN Pharmaceuticals), meperidine (e.g., DEMEROL from Sanofi Pharmaceuticals), methadone (e.g., METHADOSE from Mallinckrodt; and DOLOPHINE HCI from Roxane Laboratories), morphine (e.g., KADIAN from Faulding Laboratories, MS CONTIN from Purdue Frederick; ORAMORPH SR from Roxane), oxycodone (e.g., PERCOCET and PERCODAN from Endo; OXYCET from Mallinckrodt; OXYCONTIN from Purdue Frederick; TYLOX from Ortho-McNeil Pharmaceutical; ROXICODONE, ROXILOX and ROXICET from Roxane), pentazocine (e.g., TALACEN and TALWIN from Sanofi Pharmaceuticals), propoxyphene (e.g., DARVOCET-N and DARVON from Eli Lilly & Co.; DOLENE from Lederle; WYGESIC from Wyeth-Ayerst), and tramadol (e.g., ULTRAM from Ortho-McNeil Pharmaceutical).

Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., methyl or propyl-p-hydroxybenzoates or sorbic acid). Liquid dose forms for human administration include: hydrocodone (e.g., HYDROPHANE) from Halsey), hydromorphone (e.g., DILAUDID from Knoll), meperidine (e.g., DEMEROL from Sanofi), methadone (e.g., DOLOPHINE from Roxane), oxycodone (e.g., HYCOMINE from Knoll; ROXILOX from Roxane), and propoxyphene (e.g., DARVON-N from Eli Lilly).

For parenteral administration, including intravenous, intramuscular, or subcutaneous administration, the compounds may be combined with a sterile aqueous solution which is preferably isotonic with the blood of the recipient. Such formulations may be prepared by dissolving solid active ingredient in water containing physiologically compatible substances such as sodium chloride, glycine, or the like, and/or having a buffered pH compatible with physiological conditions to produce an aqueous solution, and/or rendering said solution sterile. The formulations may be present in unit dose forms or multi-dose forms, including in containers such as sealed ampoules or vials. Parenteral dose forms for human administration include: alfentanil (e.g., ALFENTA from Akom), buprenorphine (e.g., BUPRENEX from Reckitt & Colman Pharmaceuticals), butorphanol (e.g., STADOL from Apothecon), dezocine (e.g., DALGAN from Astrazeneca), fentanyl, hydromorphone (e.g., DILAUDID-HP from Knoll), levallorphan (e.g., LORFAN from Roche), levorphanol (e.g., LEVO-DROMORAN from ICN), meperidine (e.g., DEMEROL from Sanofi), methadone (e.g., DOLOPHINE HCI from Roxane), morphine (e.g., ASTRAMORPH from Astrazeneca; DURAMORPH and INTMORPH from Elkins-Sinn), oxymorphone (e.g., NUMORPHAN from Endo), nalburphine (e.g., NUBAIN from Endo Pharmaceutical), and pentazocine (TALWIN from Abbott).

For transdermal administration, the compounds may be combined with skin penetration enhancers such as propylene glycol, polyethylene glycol, isopropanol, ethanol, oleic acid, N-methylpyrrolidone, or the like, which increase the permeability of the skin to the compounds, and permit the compounds to penetrate through the skin and into the bloodstream. The compound/enhancer compositions also may be combined additionally with a polymeric substance such as ethylcellulose, hydroxypropyl cellulose, ethylene/vinylacetate, polyvinyl pyrrolidone, or the like, to provide the composition in gel form, which can be dissolved in solvent such as methylene chloride, evaporated to the desired viscosity, and then applied to backing material to provide a patch. Transdermal dose forms for human administration include fentanyl (e.g., DURAGESIC from Janssen).

Additional dose forms available as suppositories for human administration include oxymorphone (e.g., NUMORPHAN) from Endo).

### EXAMPLES

### Example 1

### Colorectal Distension Assay

Sensitivity for pain was analyzed using the colorectal distension assay (CRD) as described by Sawyer et al., J Amer Hosp Assoc 1987; 23: 438-46 (1987). For the studies described herein, male Sprague-Dawley rats (about 220 rats) weighing 300 to 500 grams were approved for use by the All-University Committee on Animal Use and Care of Michigan State University in accordance with NIH standards. All animals were trained over a two-month period to adjust to insertion of a lubricated (KY Jelly, Skillman, N.J., USA) colonic balloon-catheter (Pointe Medical, Crown Point, Ind., USA) via the rectum. Subjects were preconditioned to lie quietly in a towel wrapped snugly around them and tolerate the catheter in place over extended periods. The animals were offered “treats” and subsequent “play and socializing time” on a large table top with cage mates among towels, boxes and tubes (“toys”) in order to reduce the stress induced by the testing paradigms. These play periods were interspersed between testing periods for one to two hours per interval.

### Drugs

The opiate receptor agonists used in the studies are set out in Table 1. Agonists were dissolved in saline solution. Drugs and saline were injected subcutaneously (SC) via separate syringes at different sites under the skin of the back of the neck.

TABLE 1

| Drug | Type of Agonist | Source |
|---|---|---|
| Fentanyl | mu-opioid receptor | Elkins-Sims, Inc., Cherry Hill, |
| citrate (F) | selective agonist | NJ, USA |
| Spiradoline | kappa-opioid receptor | Supplied by Dr. P. L. von |
| | selective agonist | Voigtlander, Upjohn, |
| | | Kalamazoo, MI, USA |
| Enadoline | kappa-opioid receptor | Supplied by Dr. David Downs, |
| | selective agonist | Parke-Davis Pharmaceutical |
| | | Research, Ann Arbor, MI |
| Oxymorphone | mu-opioid receptor | Mallinckrodt, Mundelein, IL |
| | selective agonist | |

The opioid antagonists beta-funaltrexamine (β-FNA) and nor-binaltorphimine (n-BNI) were dissolved in sterile water and were supplied by the National Institute on Drug Abuse, Bethesda, Md., USA. Drugs and saline were injected in separate injections (not separate syringes) by the subcutaneous route (SC).

### Nociceptive Stimulus Equipment and Parameters

The CRD assay was used to establish the nociceptive thresholds (pain thresholds). In the CRD assay, air-pressure pulse-stimuli were administered to restrained subjects through inflation of a balloon-catheter. To insure a standard reproducible brief stimulus, a stimulus-pulse shaper was devised, which consisted of a 4-liter glass-jar reservoir fitted with tubing and three-way stop-cocks yoked to the jar, the catheter, a sphygmomanometer, a bicycle pump, and a port to room air. The reservoir was charged with a pressure-head between 40 and 180 mm Hg to accommodate sub-threshold, threshold, and antinociceptive responses. The nociceptive stimulus was delivered by opening the line from the reservoir to the catheter (placed within the subject's rectum), then from catheter to the open air over a maximal period of one second. Thus, at least 6 stimuli could be delivered over the span of one minute. Two stimuli were delivered within 10 seconds, yielding essentially identical signals (or lack of), to establish a valid response.

Initial lower sub-threshold pressure-pulses, frequently and randomly presented, extinguished incidental conditioning. When a threshold pulse or greater was delivered, the rat responded with an abdominal contraction (“guarding reflex”). This nociceptive response was measured via a water-filled doughnut, Disposa-Cuff (Critikon, Tampa, Fla.), fitted around the subject's abdomen. Tubing from the Disposa-Cuff to a pressure transducer relayed the signal to a polygraph recorder (Grass Instruments, Quincy, Mass.). The maximal amplitude of pressure pulses was restricted to avoid any potential tissue damage.

### Dose-Response Determinations of Agonists

After a nociceptive threshold was determined, the balloon-catheter was removed and the rat was released from the towel to be injected subcutaneously with a coded (researcher blinded) drug or placebo, using mild hand restraint. The subject was then towel-restrained again for nociceptive testing at 15-minute intervals for 30 minutes or as long as three hours post-injection. Subjects that had been tested only with single drugs were used again (no more than 3 times) in later tests, but only after a minimum of a week and after three daily typical threshold (placebo) responses. The opioid agonists were first tested alone for log-dose patterns of antinociception for fentanyl, spiradoline, enadoline, and oxymorphone. The details and results of this experiment are provided in Example 2 below.

The theoretical additive effect of single doses of agonist pairs were compared with the actual effects of combined agonist pairs using the following protocol. The single dose levels of agonists that produced antinociceptive effects that ranged from approximately 20-50% maximal percentage effects (see below) were combined and tested for the actual combined-agonist scores. Statistical comparisons of the theoretical and actual scores, the method for which is described below, established the additive, sub-additive or supra-additive differences of the actual combined-agonist interactions relative to their single dose effects. Details and results of this study are provided in Example 2 below.

Two additional tests of combined fentanyl-spiradoline and fentanyl-enadoline were conducted to determine antinociceptive interactions at 15 and 30 minutes post-injection for a high and low dose level. This study is described in detail in Example 3.

### Selective Agonist-Antagonist Determinations

Prior to testing, one Set of three groups of rats were pretreated with saline for 24 or 48 hours before testing. Another Set of three groups of rats were pretreated with 8 mg/kg β-FNA (mu-selective antagonist, Ward et al., J. Pharmacol. Exp. Ther. 220: 494-8 (1982) for 24 hrs before testing. A third Set of three rats were pretreated with 10 mg/kg n-BNI (kappa-selective antagonist, Jones et al., Eur. J. Pharmacol. 215: 345-8 (1998) for 48 hours before testing. Subsequently, all 3 Sets received 0.012 mg/kg fentanyl, 0.3 mg/kg spiradoline, or the combination, and were tested for nociceptive threshold 15 and 30 minutes later. Table 2 lists n values for groups and depicts a grid of treatments these subjects received.

TABLE 2
Number of Subjects, Pretreatment and Treatment Conditions for Grid
Testing Agonist-Antagonist Interactions in CRD

| Set I: Saline | | | Set II: β-FNA^{(a)} | | | Set III: nor-BNI^{(b)} | | |
|---|---|---|---|---|---|---|---|---|
| pretreatment | | | pretreatment | | | pretreatment | | |
| F | Sp^{(d)} | C^{(e)} | F | Sp | C | F | Sp | C |
| 8 rats | 8 rats | 10 rats | 4 rats | 4 rats | 6 rats | 4 rats | 4 rats | 6 rats |

^{(a)}beta-Funaltrexone, 8 mg/kg SC, 24 hrs before test
^{(b)}nor-Binaltorphimine, 10 mg/kg SC, 48 hrs before test
^{(c)}F = fentanyl 0.012 mg/kg
^{(d)}Sp = spiradoline 0.3 mg/kg
^{(e)}C = combined agonists

### Example 2

### Antinociceptive Responses of Combinations of Opioid Agonist

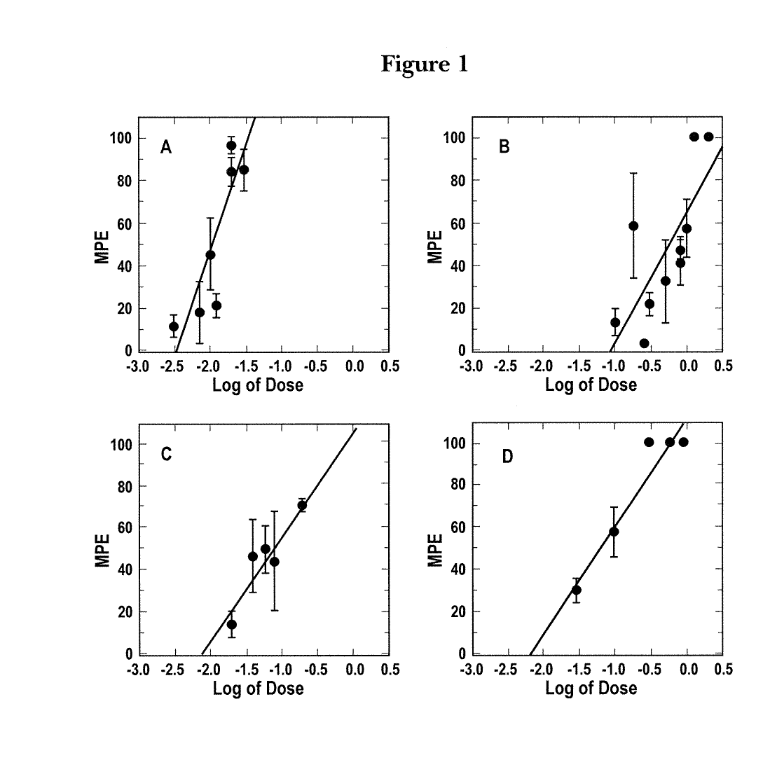

To analyze the antinociception (ANC) for several opioid agonists, the theoretical sums of these agonists in the CRD assay were determined. Individual mean log-dose-response patterns (±SEM) in the CRD assay for fentanyl, spiradoline, enadoline, and oxymorphone formed linear slopes ranging from just significant to full antinociception (ANC) with little deviation (FIG. 1). Fentanyl duration was 50 minutes with an ED₅₀ of 0.01 mg/kg (range: 0.06-0.016) and a peak effect at 15 minutes post injection (FIG. 1A). Spiradoline duration was 2 hours with an ED₅₀ of 0.56 mg/kg (range: 0.25-1.26) and a peak effect at 15 minutes post injection (FIG. 1B). Oxymorphone and enadoline served as typical-class reference comparisons. Enadoline (kappa-opioid receptor agonist)had a ED₅₀=0.077 (0.04-0.2) and a peak effect at 30 minutes post-injection (FIG. 1C). Oxymorphone (mu-opioid receptor agonist) had an ED₅₀=0.078 (0.02-0.126) and a peak effect at 30 minutes post-injection (FIG. 1D).

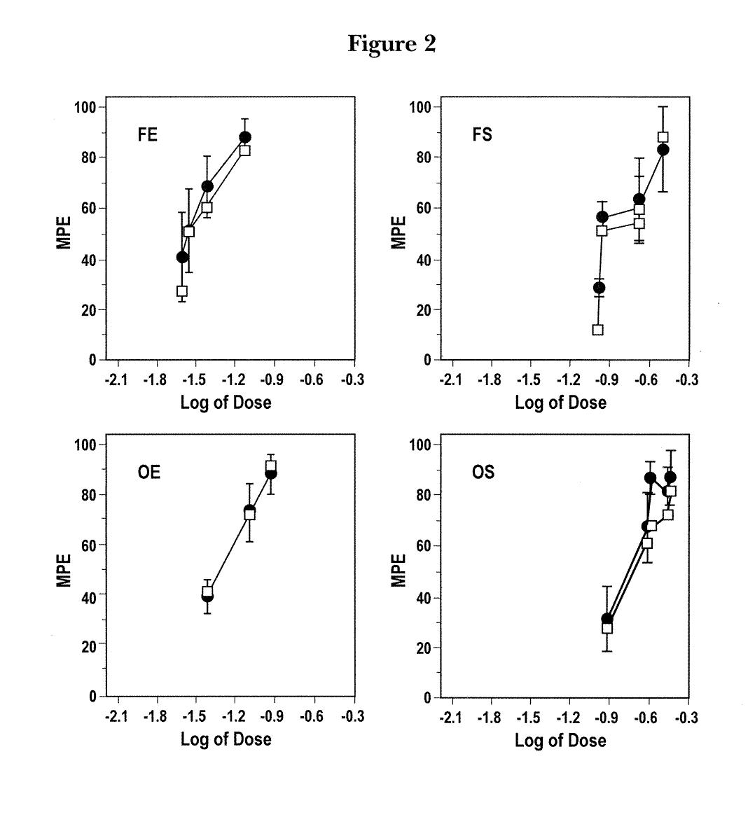

Subsequently, the actual responses of the drug combinations were compared to their theoretical sums set out in FIG. 1. At 15 minutes post-injection, the results indicated mostly additive ANC interactions, with one exception (FIG. 2). The exception was one point of actual combined-dose values of oxymorphone plus spiradoline (FIG. 2D), which yielded a supra-additive (synergistic) effect. Otherwise the actual combined effects of the 4 agonist pairs (singly scoring 20-50% Maximum Percentage Analgesic Effect (M.P.E.)) formed fairly linear slopes not significantly different from the theoretical slopes of added single doses at 15 minutes post-injection. The data in FIG. 2 is displayed as log dose of each drug, combined to test additive and potential synergistic action. The log ratios allow for comparisons between the theoretical and actual combined dose values.

An M.P.E. is the maximum percentage analgesic effect of a designated dose. For example, fentanyl at a dose of 0.02 mg/kg would produce near-maximal analgesia (M.P.E.=90%+) in the CWTF. An ED-50 dose would be about 0.009 mg/kg (M.P.E.=50%), and an ED-10 would likely be 0.003 mg/kg (M.P.E.=10%).

The results of low- and high-dose combinations of fentanyl plus spiradoline and fentanyl plus enadoline, tested for ANC at 15 minutes and 30 minutes post-injection, are shown in FIG. 3.

Full ANC levels for either class of opioid agonist were observed in the cold-water tail-flick assay (CWTF) as described in Briggs et al., Pharmacol. Biochem. Behav. 60: 467-72 (1998). However, the dose-effect pattern of ANC for the combination in the CWTF assay differed from that in the CRD assay described herein. In CWTF, low-dose combinations produced additive effects, while high-dose combinations produced sub-additive or antagonistic interactions. In CRD, low doses in combination induced additive effects and the combination of high doses resulted in supra-additive ANC patterns.

In FIG. 3 (left-hand panel A), ANC of the low-dose fentanyl was greater than that of the higher-dose fentanyl response (right-hand panel A). This anomaly may relate to the repeated testing of the subjects (maximum of 3 treatments) with single doses of opioid receptor agonists, even though these treatments were spaced a week apart and at least 3 days of placebo tests were carried out between treatments. Pearl et al., Neurosci. Lett. 213: 5-8 (1996) reported interactions of U50,488H or spiradoline with morphine, reducing morphine enhancement of locomotor activity when morphine was injected 19 hours after administration of either kappa-opioid receptor agonist. The kappa-opioid receptor antagonism was further strengthened by 2 days of morphine pretreatment. Thus, mu- and kappa-receptor opioid agonistic influences on neuroplasticity appear to far outlast (45 hours or more) the usual ANC duration of single-dose effects.

### Example 3

### Antinociceptive Responses for Combined Agonists and Antagonists

The high-dose combination of fentanyl plus spiradoline resulted in supra-additive interactions at both time periods tested (15 and 30 minutes post injection, FIG. 3 left-hand panel A). Tests of the other dose combinations formed additive response patterns. The single low dose of fentanyl in panel A scored a higher M.P.E. (45, 15 minutes) than the single high dose of fentanyl (18, 15 minutes). Fentanyl “freezing” behavior (catalepsy) was not observed in this study. FIG. 4 presents the single antinociceptive-dose effects of fentanyl (0.012 mg/kg), spiradoline (0.3 mg/kg), and the combined-dose effects of opioid receptor agonists after saline pretreatment, beta-funeltrexamine (β-FNA) pretreatment, or nor-binaltorphimine (n-BNI) pretreatment in three “Sets” of rats (9 groups in all).

As shown in FIG. 4A, the combination of fentanyl and spiradoline after saline pretreatment induced a significantly greater analgesic effect, as measured by the Maximal Percentage Analgesic Effect (M.P.E), than fentanyl alone (*, p<0.01), or spiradoline alone (*, p<0.01) 15 minutes after injection. Pretreatment with an opioid receptor antagonist (β-FNA or nor-BNI) did not affect the analgesic effect of fentanyl alone compared to fentanyl pretreated with saline. The analgesic effect of spiradoline was significantly reduced with β-BFA pretreatment when compared to spiradoline pretreated with saline (#, p<0.01). The analgesic effect of the combination of fentanyl and spiradoline, after pretreatment with β-FNA or nor-BNI, was significantly greater than the analgesic effect induced by fentanyl alone or spiradoline alone with saline pretreatment (* p<0.05). However, the analgesic effect of the combination after pretreatment with an opioid receptor antagonist was significantly reduced compared to the effect of the combination with saline pretreatment (@, p<0.01). These data demonstrate that the combination of mu- and kappa-opioid receptor agonists in the presence of an ultra-low dose of an opioid receptor antagonist induces a greater analgesic effect than administration of an opioid receptor agonist alone.

As shown in FIG. 4B, at thirty-minutes post-injection, the analgesic effect of the combination of fentanyl and spiradoline after saline pretreatment was significantly reduced compared to the analgesic effect induced by the combination 15 minutes post-injection shown in Panel A (** p<0.01). However, the analgesic effect induced by the combination 30 minutes post-injection was significantly greater then any of analgesic effects of the agonist alone or in combination after pretreatment with opioid receptor antagonists (* p<0.01).

After saline pretreatment, both fentanyl and spiradoline individually produced an approximate ED₂₀ ANC response at the 15-minute test period (mean M.P.E. for fentanyl=21% and for spiradoline=22%). The drug combination after saline pretreatment induced prominent synergistic ANC (mean M.P.E. for C=68%). At the 30-minute test, the combined agonists continued to manifest a supra-additive effect in the saline-pretreatment group (mean M.P.E.=38%), compared to the mean single-dose fentanyl score of 14% and the mean single-dose spiradoline score of 3%.

Surprisingly, the fentanyl M.P.E. score was not reduced after β-FNA pretreatment compared to that of the saline-pretreatment group (30% vs. 21%) at the 15-minute test period. The spiradoline M.P.E. was significantly decreased (4% vs. 22%) after β-FNA in this period. The combined agonists after β-FNA resulted in a score significantly reduced (33%) compared to the combined agonists' score in the saline-pretreatment group (68%).

The n-BNI pretreatment failed to significantly alter the individual agonist scores at either the 15- or 30-minute test periods compared to those of saline controls. However, the score of the combined drugs after n-BNI was much lower compared to those of saline-pretreatment rats (18% vs. 68% at the 15-minute test, and 13% vs. 38% at the 30-minute test).

To emphasize the difference of the paradoxical effects in FIG. 4 compared to the agonist-antagonist interactions found in the CWTF assay (Briggs et al., Pharmacol. Biochem. Behav. 60: 467-72 (1998), the results from the CWTF study are repeated in Table 3. Using the CWTF assay, the mean M.P.E. of fentanyl was 86% and that of spiradoline was 77% after saline pretreatment. After β-FNA pretreatment the fentanyl score was significantly reduced to 21%. The spiradoline score was a non-significant decrease to 67%. After n-BNI pretreatment, the fentanyl score was 73% and the spiradoline score was significantly reduced to 13%.

TABLE 3
Antinociception of Fentanyl (F) and Spiradoline (Sp) Using the Cold-Water Tail-Flick
Assay in Saline-, β-FNA-, or n-BNI-Pretreated Rats. (From Briggs et al.,
Pharmacol. Biochem. Behav. 60: 467-72 (1998)

| | Saline (a) | | | β-FNA (a) | | | n-BNI (b) | | |
|---|---|---|---|---|---|---|---|---|---|
| | S | F | Sp | S | F | Sp | S | F | Sp |
| M.P.E. | 1.0 | 86 +/− 6 | 77 +/− 8 | 1.0 | 21 +/− 10 | 67 +/− 12 | 1.0 | 73 +/− 9 | 13 +/− 5 |

(a) Saline or beta-funaltrexone (β-FNA), 8 mg/kg, pretreatment was injected SC 24 hrs before testing.
(b) nor-Binaltorphimine (n-BNI), 10 mg/kg, was injected SC 48 hrs before testing.
(c) Maximal percent effect, antinociceptive values are means +/− S.E.M., n = 6 for each group.
(d) Values of F and Sp differ significantly, p < 0.01, from F and Sp after saline pretreatment, respectively.
S (saline),
F (fentanyl, 0.018 mg/kg), or
Sp (spiradoline, 1.0 mg/kg), was injected SC 15 min prior to antinociceptive testing.

Regarding agonist-antagonist interactions (fentanyl plus spiradoline, β-FNA plus n-BNI), prior results in the CWTF assay (discussed above) were straightforward. β-FNA (mu-selective antagonist) markedly decreased the ANC of fentanyl without a significant change in spiradoline ANC. After n-BNI (kappa-specific antagonist), a reduced ANC of spiradoline (selective kappa-opioid receptor agonist) occurred, while no significant change in the ANC of fentanyl was observed. However, agonist-antagonist interactions in CRD (FIG. 4) resulted in paradoxical reactions. After β-FNA, fentanyl ANC-CRD tended to increase (non-significantly) while spiradoline ANC was attenuated, relating to individual agonist effects in saline-pretreatment subjects. After n-BNI, neither fentanyl nor spiradoline single-dose ANC was significantly altered from those of the saline-pretreated subjects. The use of low ANC dose levels of the agonists in the CRD tests (M.P.E. of approximately 20%) may have compromised the extent of antagonism rather than optimize synergistic ANC interactions of the two agonists as intended. Other possible explanations for such complex opioid interactions are discussed below.

It is theorized that failure of β-FNA pretreatment to alter ANC of fentanyl in the CRD (as shown in FIG. 4) could occur by several mechanisms. One possible mechanism is that a supra-spinally or spinally innervated mu-opioid receptor link may exert tonic inhibitory control over spinal kappa-opioid-agonist mechanisms, resulting in a blockade of the mu-opioid receptors by β-FNA. The blocking of the mu-receptors then could result in disinhibition of the spinal kappa mechanism, and ANC would be induced by release of an endogenous kappa-opioid receptor agonist. Likewise, the decreased ANC of spiradoline after β-FNA could relate to chronic supra-spinal or spinal kappa-opioid mechanisms activating release of an endogenous mu-opioid agonist. The resulting mu-opioid receptor agonist then would inhibit spinal pain-projection neurons reacting to incoming distal nociceptive stimuli. Spiradoline would still release endogenous mu-opioid receptor agonist, but β-FNA blockade of post-junctional mu-opioid receptors would attenuate the ANC response.

The interactions described above are consistent with the synergism of ANC by combined agonists in the saline-pretreatment group (FIG. 4) being decreased after either antagonist pretreatment, β-FNA or n-BNI. The greater antagonism by n-BNI of the combined agonist ANC synergy may indicate (as suggested by Schmauss et al., Eur. J. Pharmacol. 135: 429-31 (1987) a dominant role of kappa-opioid receptor mechanisms in the suppression of visceral pain. Staahl et al., Pain. 123: 28-36 (2006) showed oxycodone to induce superior ANC vs. morphine in human subjects exposed to experimental visceral nociception. Since oxycodone is a kappa-opioid receptor agonist metabolized to a mu-opioid receptor agonist (Ross et al., Pain. 84: 421-8 (2000), these results imply a combined mu- and kappa-opioid interaction.

Interactions between exogenous mu- and kappa-opioids, as well as those between endogenous opioids, seem to be most implicated in conditions involving chronic visceral pain. Several clinically-oriented reviews have promoted the concept of employing opioid drug combinations for improved therapeutic management of pain while reducing adverse drug side effects (Coop Amer. J. Pharm. Educ. 24: 198-205 (2002); Smith, Pain Physician. 11: 201-14 (2008). Joshi et al., Curr. Rev. Pain. 4: 499-506 (2000) reviewed the need for greater knowledge and research in the areas of visceral pain and for candidate opioid and non-opioid therapies. This review emphasized the discovery of the dorsal column pain pathway, further integrating spinal and supraspinal nociceptive and ANC mechanisms, thus identifying new sites at which drugs may interact to modulate visceral pain mechanisms. Extensive research on this topic would likely aid in the development of more effective therapies.

### Example 4

### Fentanyl and Spiradoline Interactions for Place Conditioning Responses in a Black-White Shuttle-Box

The following study demonstrates that the kappa-opioid receptor agonist-induced adverse side effect dysphoria can be counteracted by activation of mu-opioid receptors, and fentanyl-induced euphoria is reciprocally counteracted by activation of kappa-opioid receptors.

Male Sprague-Dawley adult rats, weighing 220 g to 350 g, were divided into four groups of 6 each (denoted as Groups A, B, C, and D). The rats were trained for place preference and place aversion to the selective mu-opioid agonist, fentanyl, and the selective kappa1-opioid agonist, spiradoline, respectively. Group A received only saline subcutaneous injections (placebo) throughout the study. Group B was trained and tested on three dose levels of fentanyl before testing, then trained on combined agonists before the last test. Group C was trained on two doses of fentanyl before Tests 1 and 2, then trained on two doses of spiradoline before Tests 3 and 4, and finally trained on combined agonists before the last test. Group D was trained first on three dose levels of spiradoline before testing, then trained on the combined agonists before Test 5.

The drug dose levels were chosen from antinociceptive dose-response patterns of previous studies using the CWTF and CRD assays (Briggs et al., Pharmacol. Biochem. Behav. 60: 467-72 (1998); Briggs and Rech, 2008). Fentanyl citrate (Elkin-Sims, Inc., Cherry Hill, N.J., USA) doses for the current study were 0.003, 0.006, and 0.012 mg/kg. Spiradoline doses were 0.3, 0.6, and 1.2 mg/kg (Spiradoline was generously provided by Dr. P. L. von Voigtlander, Upjohn Co., Kalamazoo, Mich., USA). Drugs were dissolved in normal saline and administered by subcutaneous (SC) injection. Fentanyl was injected 15 minutes before placing subjects into the shuttle box, and spiradoline was injected 30 minutes before placing subjects into the shuttle-box. The pretreatment times were based upon peak antinociceptive activities. The animals and procedures were approved for this study, conforming to NIH standards, by the Michigan State University Animal Use and Care Committee.

### Place Conditioning Apparatus, Training and Testing Parameters

Two shuttle-boxes were constructed with two compartments each, 35 cm long×13 cm wide×13 cm high, joined at the narrow walls on one side, in which 7 cm circles were cut one centimeter above the floors. In each box, one compartment was painted black with a mesh floor and the other compartment was painted white with a smooth floor. A rectangular baffle-plate, black on one side and white on the other, was inserted between the connecting walls to restrict a rat to one or the other compartment during training. On test days the baffle was removed to allow subjects free access to both compartments. An axle fitted under the baffle-plate slot caused the box to tilt a few millimeters in the long dimension over the baseboard as a rat moved from one side to the other. This action activated or deactivated a micro-switch installed at one end of the shuttle-box. The micro-switch contacts were connected to an electric timer-event recorder, which registered the times of tilts by a needle displacement running on pressure-sensitive paper tape. Thus, the percentage of a 15-minute period that the subject spent in each compartment during a test session was determined by reading the tapes.

Three of the 6 rats in each group were restricted to the black compartment and the other three restricted to the white compartment on drug-training days. For each subject, this compartment was designated the “drug-associated compartment.” On placebo-training days subjects received subcutaneous injections of saline and were restricted to the opposite color compartment (“placebo-associated compartment”). All groups were initially exposed to the Pre-training Session, during which they received only saline injections to assess the biased or unbiased nature of the conditioning procedure. Group A received only saline during drug-training days and placebo-training days, so that for them the term “drug-associated compartment” was a misnomer. However, the designation was retained with Group A for the sake of conformity.

Table 4 lists pre-training, training, and testing schedules for all days, groups and treatments.

TABLE 4
Schedule of Training and Test Days, Drug and Placebo Sessions.

| | Days | Group A | Group B | Group C | Group D |
|---|---|---|---|---|---|
| Pre-training | 1, 2, 4, 6; | Saline | Saline | Saline | Saline |
| | DAC(a) | | | | |
| | 3, 5; PAC(b) | Saline | Saline | Saline | Saline |
| Pre-training | 7 | Saline | Saline | Saline | HDF1(e) |
| test | | | | | |
| Training | 8, 9, 11, 13; | Saline | HDF1(c) | LDF1(d) | HDS1(e) |
| Session 1 | DAC | | | | |
| | 10, 12; PAC | Saline | Saline | Saline | Saline |
| Test Day 1 | 14 | Saline | Saline | Saline | Saline |
| Training | 15, 16, 18, 20; | Saline | LDF2 | MDF1(f) | LDS1(g) |
| Session 2 | DAC | | | | |
| | 17, 19: PAC | Saline | Saline | Saline | Saline |
| Test Day 2 | 21 | Saline | Saline | Saline | Saline |
| Training | 22, 23, 25, 27; | Saline | MDF2 | LDS2 | MDS1(h) |
| Session 3 | DAC | | | | |
| | 24, 26; PAC | Saline | Saline | Saline | Saline |
| Test Day 3 | 28 | Saline | Saline | Saline | Saline |
| Training | 29, 30, 32, 34, | Saline | HDF2 | MDS2 | HDS2 |
| Session 4 | DAC | | | | |
| | 31, 33; PAC | Saline | Saline | Saline | Saline |
| Test Day 4 | 35 | Saline | Saline | Saline | Saline |
| Training | 36, 37, 39, 41; | Saline | HDF + LDS | MDS + MDF | HDS + LDF |
| Session 5 | DAC | | | | |
| | 38, 40; PAC | Saline | Saline | Saline | Saline |
| Test Day 5 | 42 | Saline | Saline | Saline | Saline |

(a)Subjects restricted to drug-associated compartment (DAC)
(b)Subjects restricted to placebo-associated compartment (PAC)
(c)High-dose fentanyl; 1 = first dosing; 2 = second dosing, etc.
(d)Low-dose fentanyl
(e)High-dose spiradoline
(f)Medium-dose fentanyl
(g)Low-dose spiradoline
(h)Medium-dose spiradoline

### Analysis of Drug Effects

The percent of time spent in the drug-associated compartment for the four groups during the Pre-training Test Day served as the control (placebo) scores for all drug-treatment effects. These 24 scores ranged from 47.8 to 52%, only one score falling outside the span of 48-52%. Group A scores from the following five test days ranged from 48-52%, excepting two that were slightly below 48 and one that was slightly above 52. Therefore, this place-conditioning assay was unbiased, without significant differences among groups in test sessions after saline treatments.

The data was analyzed using a computer-based program of statistical analysis. A multiple repeated measures of ANOVA generated an overall significant difference of p<0.0001, including means±standard errors (SEM) of scores for each group, allowing for within group and between group comparisons. The Tukey-Kramer Multiple Comparisons Test was then applied to all individual pairs of scores for all treatment tests, excepting the scores from Group A, during the test days. Values of q from the comparisons of pairs greater that 5.143 indicated a significance of p<0.05. A total of 171 pairs were analyzed, 127 of which were significantly different at p<0.001, (q values exceeding 7). Of the remaining 44 pairs, 30 were not significantly different, 3 differed by p<0.01, and 11 differed by p<0.05. Since the q values for 64 comparisons exceeded 20, it is obvious that those comparisons differed by greater than p<0.001, but the program did not supply the exact p values.

The sequence of drug treatments among groups, as shown in Table 4, was based upon the following strategies. In Group B, a fentanyl dose-response analysis was established, after which combined fentanyl and spiradoline was tested to examine the extent of spiradoline alteration of the level of fentanyl place conditioning. Group C was exposed to two doses of fentanyl, then two doses of spiradoline, and finally the medium doses of fentanyl plus spiradoline were combined, to assess the relative strengths of fentanyl preference vs. spiradoline aversion. Group D was trained over sessions 1-4 to establish a dose-response pattern of spiradoline aversion, then was trained on spiradoline plus fentanyl to determine the level of altered place conditioning due to combining spiradoline with fentanyl.

The results (means±SEM) obtained for fentanyl, spiradoline, and their interactions for all drug-treated groups in the place-conditioning sequence projected in Table 4 are presented in Table 5

TABLE 5

| Pre-Training | | Scores from Training Tests | | | | |
|---|---|---|---|---|---|---|
| Group | Scores | Test 1 | Test 2 | Test 3 | Test 4 | Test 5 |
| A | 49.53 (+/−1.00) | (48-52) (a) | (48-52) | (48-52) | (48-52) | (48-52) |
| B | 49.96 (+/−1.64) | HDF1 (b) | LDF2 | MDF2 | HDF2 | HDF + LDS |
| | | 74.07 | 62.63 | 71.38 | 79.67 | 68.28 |
| | | (+/−1.17) | (+/−0.98) | (+/−1.49) | (+/−1.69) | (+/−1.23) |
| C | 50.05 (+/−1.99) | LDF1 (c) | MDF1 (e) | LDS2 | MDS2 | MDS + MDF |
| | | 60.93 | 74.32 | 44.03 | 38.38 | 45.56 |
| | | (+/−0.72) | (+/−1.91) | (+/−0.77) | (+/−0.97) | (+/−1.18) |
| D | 50.27 (+/−1.45) | HDS1 (d) | LDS1 (f) | MDS1 (g) | HDS2 | HDS + LDF |
| | | 34.67 | 45.28 | 39.58 | 29.10 | 37.38 |
| | | (+/−0.87) | (+/−1.15) | (+/−0.60) | (+/−0.87) | (+/−1.01) |

(a) Range of scores for group A during Training/Test Sessions.
(b) High-dose fentanyl; HDF1 = first test of this dose; HDF2 = second test of this dose; etc.
(c) LDF = Low-dose fentanyl.
(d) HDS = High-dose spiradoline.
(e) MDF = Medium-dose fentanyl.
(f) LDS = Low-dose spiradoline.
(g) MDS = Medium-dose spiradoline.

The significant differences of 127 pairs of scores at p<0.001, from a total of 171 pairs, clearly support dose-related conditioned preference of fentanyl and dose-related conditioned aversion of spiradoline. Regarding the 30 pairs of scores lacking significant differences, most are easily justified. Six related to saline vs. saline comparisons. Others were Saline vs. MDS+MDF, LDF1 vs. LDF2, MDS vs. HDS+LDF, LDS1 vs. LDS2, MDF vs. HDF+LDS, etc., for eleven more.

The remaining thirteen non-significant comparisons were pairs with one-step dose differences: Saline vs. LDS, HDF vs. MDF, LDS vs. MDS+MDF, LDS vs. MDS, MDS vs. HDS, and HDS vs. HDS+LDF. Some of these last comparisons were likely skewed by the sequence of conditioning training. For example, consider the preference score after HDF1 (Group B initial training) being non-significant from the score after MDF2 (treatment of Group B on the third sequence of training). In addition, the initial training of Group D with HDS1 (first training session) that induced an aversive score not significantly different from the score after MDS2 exposure of Group C on the fourth sequence of training. Group C had been exposed to LDS2 during the third training session. Similar one-step dose-level differences in scores were found among most of the pairs differing in significance at p<0.01 and p<0.05.

The results support dose-related place preference and dose-related place aversion for fentanyl and spiradoline, respectively. Interactions of the two opioid agonists demonstrated suppression of fentanyl preference by spiradoline and decreased aversion from spiradoline conditioning by the combination with fentanyl. With Group B comparisons, the HDF1 score (74.07) vs. the HDF+LDS score (68.28) differed significantly at p<0.05, and the HDF2 score (79.67) vs. the HDF+LDS score differed by p<0.001. Thus, the lowest dose of spiradoline antagonized the prominent preference of the highest dose of fentanyl.

Comparing Group C MDF1 score (74.32) with the group C MDS+MDF score (45.56) resulted in a significance of p<0.001, suggesting a dominance of the motivational effect of spiradoline over the preference conditioning of fentanyl. Relating to spiradoline-induced conditioned place aversion, comparing Group D HDS2 score (29.10) with Group C HDS+LDF score (37.38), spiradoline-induced conditioned aversion was attenuated by adding the low dose of fentanyl, at a significance of p<0.001. Fentanyl's interference with the expression of spiradoline-induced conditioned aversion was again evident by comparing Group C MDS2 score (38.38) with that group's MDS+MDF score (45.56), significantly different at p<0.05. These results are consistent with somewhat reciprocal relationships for interactions of the agonists in inducing these opposing motivational states.

Numerous modifications and variations in the practice of the invention are expected to occur to those skilled in the art upon consideration of the presently preferred embodiments thereof. Consequently, the only limitations which should be placed upon the scope of the invention are those which appear in the appended claims.

1. A method of treating pain in a subject, the method comprising:
administering to a subject suffering from pain a moderate dose of a selective mu-opioid receptor agonist, a moderate dose of a selective kappa1-opioid receptor agonist, and an ultra-low dose of a nonselective opioid receptor antagonist,
wherein the doses are effective in combination to promote analgesia in the subject and to reduce an adverse side effect of pain treatment with an opioid receptor agonist in the subject.

2. A method of enhancing analgesia with an opioid receptor agonist while reducing an adverse side effect of pain treatment with an opioid receptor agonist, the method comprising:
administering to a subject suffering from pain a moderate dose of a selective mu-opioid receptor agonist, a moderate dose of a selective kappa1-opioid receptor agonist, and an ultra-low dose of a nonselective opioid receptor antagonist,
wherein the doses are effective in combination to promote analgesia in the subject and to reduce an adverse side effect of pain treatment with an opioid receptor agonist in the subject.

3. The method of claim 1, wherein the doses are effective in combination to provide enhanced analgesia compared to analgesia from a moderate dose of either of said opioid receptor agonists alone.

4-11. (canceled)

12. The method of claim 1, wherein the selective mu-opioid receptor agonist is selected from the group consisting of oxymorphone, hydrocodone, hydromorphone, levorphanol, oxycodone, methadone and fentanyl.

13-19. (canceled)

20. The method of claim 1, wherein the selective kappa1-opioid receptor agonist is a arylacetamide opioid receptor agonist.

21. The method of any one of claims 19, wherein the selective kappa1-opioid receptor agonist is spiradoline or enadoline.

22-23. (canceled)

24. The method of claim 1, wherein the selective mu-opioid receptor agonist is fentanyl and the selective kappa1-opioid receptor agonist is spiradoline.

25. The method of claim 1, wherein the selective mu-opioid receptor agonist is oxymorphone and the selective kappa1-opioid receptor agonist is spiradoline.

26. The method of claim 1, wherein the selective mu-opioid receptor agonist is fentanyl and the selective kappa1 agonist is enadoline.

27. The method of claim 1, wherein the selective mu-opioid receptor agonist is oxymorphone and the selective kappa1 agonist is enadoline.

28. The method of claim 1, wherein the nonselective opioid receptor antagonist is selected from the group consisting of naloxone and naltrexone.

29-39. (canceled)

40. A composition comprising a moderate dose of a selective mu-opioid receptor agonist, a moderate dose of a selective kappa1-opioid receptor agonist, and an ultra-low dose of a nonselective opioid receptor antagonist, wherein the doses in combination are effective to reduce pain and to reduce an adverse side effect of treatment with an opiate receptor agonist in a subject.

41-42. (canceled)

43. The composition of claim 40, wherein the selective mu-opioid receptor agonist is selected from the group consisting of hydrocodone, hydromorphone, levorphanol, oxycodone, oxymorphone, methadone and fentyanyl.

44-50. (canceled)

51. The composition of claim 50, wherein the selective kappa1-opioid receptor agonist is an arylacetamide opioid agonist.

52. The composition of claim 51, wherein the selective kappa1-opioid receptor agonist is spiradoline or enadoline.

53-54. (canceled)

55. The composition of claim 40, wherein the selective mu-opioid receptor agonist is fentanyl and the selective kappa1-opioid receptor agonist is spiradoline.

56. The composition of claim 40, wherein the selective mu-opioid receptor agonist is oxymorphone and the selective kappa1-opioid receptor agonist is spiradoline.

57. The composition of claim 40, wherein the selective mu-opioid receptor agonist is fentanyl and the selective kappa1 agonist is enadoline.

58. The composition of claim 40, wherein the selective mu-opioid receptor agonist is oxymorphone and the selective kappa1 agonist is enadoline.

59. The composition of claim 40, wherein the nonselective opioid receptor antagonist is selected from the group consisting of naloxone and naltrexone.

60-70. (canceled)
